(19) United States
(12) Patent Application Publication
(10) Pub. No.: US 2021/0317537 A1
(43) Pub. Date: Oct. 14, 2021

(54) NOVEL MRNA BASED PROGNOSTIC INDEX DERIVED FROM DIFFERENTIAL EXPRESSION ANALYSIS IMPROVES OVERALL SURVIVAL ESTIMATES IN GLIOBLASTOMA

(71) Applicant: ARIZONA BOARD OF REGENTS ON BEHALF OF THE UNIVERSITY OF ARIZONA, Tucson, AZ (US)

(72) Inventors: Eric Weterings, Tucson, AZ (US); Baldassarre D. Stea, Tucson, AZ (US); Daruka Mahadevan, Tucson, AZ (US); Christopher Michael Morrison, Tucson, AZ (US); Michael F. Hammer, Tucson, AZ (US)

(21) Appl. No.: 17/320,739

(22) Filed: May 14, 2021

Related U.S. Application Data
(63) Continuation-in-part of application No. PCT/US19/55720, filed on Oct. 10, 2019.
(60) Provisional application No. 62/767,914, filed on Nov. 15, 2018.
(60) Provisional application No. 63/061,723, filed on Aug. 5, 2020.

(51) Int. Cl. C12Q 1/6886

(52) U.S. Cl. CPC C12Q 1/6886; C12Q 2600/106; C12Q 2600/158; C12Q 2600/156; C12Q 2600/118; C12Q 2600/154

(57) ABSTRACT

The present invention relates to diagnostic assays useful in classification of patients for selection of cancer therapy, and relates to a method of using a select family member of the Wnt signaling pathway (WIF1), an important component of the insulin-like growth factor pathway (IGFBP3), and other signaling factors (e.g., NGFR, IBSP, and HISTLH3G and COL1A2) as prognostic markers and potential therapeutic targets for patients with Glioblastoma Multiforme (GBM). In particular, the present invention is a novel GBM Prognostic Index (GPI) that predicts overall survival (OS) in GBM patients treated with the current standard of care.

### CROSS-REFERENCES TO RELATED APPLICATIONS

This application is a Continuation-In-Part and claims benefit of PCT Application No. PCT/US19/55720, filed Oct. 10, 2019, which claims benefit of U.S. Provisional Application No. 62/767,914, filed Nov. 15, 2018, the specifications of which are incorporated herein in their entirety by reference.

This application is a Non-Provisional patent application and claims benefit of U.S. Provisional Application No. 63/061,723, filed Aug. 5, 2020, the specifications of which are incorporated herein in their entirety by reference.

### FIELD OF THE INVENTION

The present invention relates to diagnostic assays useful in classification of patients for selection of cancer therapy, and in particular relates to a method of using select members of specific signaling pathways (e.g., Wnt signaling pathway) as predictive markers and therapeutic targets for patients with Glioblastoma Multiforme (GBM). In particular, the present invention is a novel GBM Prognostic Index (GPI) that predicts overall survival (OS) in GBM patients treated with the current standard of care. The GPI utilizes nine (9) factors that are significantly associated with an increased risk of death and comprises a patient's age, a unique panel of six (6) genes comprising WIF1 (Wnt inhibitory factor-1), IGFBP3 (insulin-like growth factor binding protein-3), NGFR (nerve growth factor receptor), IBSP (integrin binding sialoprotein), HIST1H3G (histone H3), and COL1A2 (collagen alpha-2) as well as IDH (isocitrate dehydrogenase 1) mutation status and MGMT (O⁶-Methylguanine-DNA Methyltransferase) methylation status.

The present invention may be used as a companion diagnostic assay (CDx) to predict radiotherapy and chemotherapy outcome in patients with GBM to better assess diagnosis, prognosis, and therapeutic strategies for each individual patient. The present invention allows for a quantitative measurement of the GPI based on the age of the patient age, on the expression level of six (6) signaling pathway markers WIF1, NGFR, IGFBP3, IBSP, HIST1H3G, and COL1A2 as well as IDH mutation status and MGMT methylation status. The levels of these markers can be determined in frozen or formalin fixed paraffin embedded (FFPE) tumor tissue, and possibly cerebrospinal fluid (CSF), and/or blood from patients with GBM using standard analytical techniques. In addition to the patient's age, this six gene profile, IDH mutation status and MGMT methylation status comprises the GPI and facilitates identification of those GBM patients who may benefit from additional therapy beyond the standard chemo-radiation (chemoRT) (e.g., enrolling on a clinical trial to test novel therapies such as a Wnt pathway inhibitor), and/or additional or more frequent monitoring. This invention is not limited to patients with GBM and may be utilized for any condition that is eligible to receive radiation or conditions with dysregulation in the identified pathways (e.g., conditions caused by or cause Wnt signaling dysfunction).

### BACKGROUND OF THE INVENTION

Glioblastoma Multiforme (GBM), a World Health Organization (WHO) grade IV astrocytoma, is the most commonly diagnosed primary brain tumor in adults. The disease is characterized by extreme invasiveness beyond the surgical bed, rapid progression, and high treatment resistance. Little progress has been made in the treatment of GBM since the NCIC/EORTC trial by Stupp et al. defined the current standard of care, which consists of maximal safe resection followed by adjuvant temozolomide (TMZ) and 60 Gy of fractionated radiation. At present, this standard of care is not curative and median overall survival (OS) for GBM is approximately 14 months. The recent addition of electrical fields (Optune, Novocure) to the Stupp regimen has shown a promising improvement in median OS by 5 months, but this treatment modality has a number of barriers to wide-spread implementation. While significant advancements have been made in the understanding of GBM pathogenesis, only a very limited number of prognostic markers and potential therapeutic targets have been identified.

In addition to established clinical prognostic factors, such as age, tumor size and location, extent of resection, and Karnofsky Performance Score (KPS), a few genetic markers are currently used to inform prognosis. Most notably, mutations in the isocitrate dehydrogenase (IDH) gene predict for longer OS, while telomerase reverse transcriptase (TERT) promoter mutations predict a shorter OS. In addition, epigenetic silencing of O⁶-methylguanine-DNA methyltransferase (MGMT) is both prognostic and a predictor of an increased response to TMZ and thus a longer OS.

Because patients can have widely different clinical courses after nearly identical treatments, the present invention undertook a retrospective genetic analysis of GBM specimens obtained from patients at the time of initial tumor resection and correlated their mRNA expression profiles to the OS of these same patients. The goal was to search for biological differences between patients with short OS versus those with relatively long OS after standard treatment with surgery, chemoRT, and adjuvant chemotherapy. Using comparative differential expression analyses combined with multivariate Cox proportional hazards (CPH) models, the present invention derives a prognostic index that can discriminate between patients with significantly different OS expectations. The utility of the prognostic index was then validated within an independent, publicly available dataset from The Cancer Genome Atlas (TCGA). The present invention demonstrates the clinical significance of these findings and proposes that a prognostic index based on the identified gene signature could be a powerful tool to support clinical decision making regarding GBM prognosis.

### BRIEF SUMMARY OF THE INVENTION

It is an objective of the present invention to provide methods that allow for predicting differential overall survival (OS) in Glioblastoma Multiforme (GBM) patients treated with the current standard of care as specified in the independent claims. Embodiments of the invention are given in the dependent claims. Embodiments of the present invention can be freely combined with each other if they are not mutually exclusive.

The present invention features a new method of using select signaling pathways in combination (e.g., Wnt signaling pathway and insulin-like growth factor signaling pathway) as well as in combination with the age of the patient, IDH mutation status and MGMT methylation status as a companion diagnostic for diagnostic, prognostic and treatment tailoring and monitoring purposes to aid in the personalized management of patients at risk of or with GBM. Physicians will be able to use this assessment to recommend personalized treatments (e.g., precision therapy), a feature commonly unavailable to patients with GBM. This technology provides a way to effectively predict differential OS of those patients who will benefit from either form of therapy (e.g., radiation, chemotherapy), effectively assisting physicians in making the best decisions for their patients and avoiding the toxic effects of either therapy.

One of the unique and inventive technical features of the present invention is using a panel of six (6) genes, WIF1 (Wnt inhibitory factor-1), IGFBP3 (insulin-like growth factor binding protein-3), NGFR (nerve growth factor receptor), IBSP (integrin binding sialoprotein), HIST1H3G (histone H3), and COL1A2 (collagen alpha-2) to assess for select signaling dysfunctions to aid personalized medicine for GBM. In particular, this invention utilizes a unique GPI (GBM prognostic index) that is a prognostic indicator for that will help inform personalized treatment strategies and disease progression monitoring for GBM and other conditions with dysregulation in the identified pathways (e.g., that cause or are caused by Wnt or insulin-like growth factor signaling dysfunction). The GPI comprises nine (9) factors: the age of the patient at diagnosis and the expression signatures (or expression profiles or disease profiles) of a panel of six (6) genes, WIF1, NGFR, IGFBP3, IBSP, HIST1H3G, and COL1A2, IDH (isocitrate dehydrogenase 1) mutation status and MGMT (O⁶-Methylguanine-DNA Methyltransferase) methylation status. Without wishing to limit the invention to any theory or mechanism, it is believed that the unique GPI of the present invention advantageously provides for personalized treatment approaches for GBM and conditions that cause or caused by Wnt or insulin-like growth factor signaling dysfunction. None of the presently known prior references or work has the unique inventive technical feature of the present invention.

The present invention is not obvious because the prior art teaches away from using the panel of identified genes as a biomarker for personalized treatment of GBM. It was surprising that this panel of 6 genes along with the other 3 clinical factors was able to discriminate between patients with significantly different OS expectations much better than the standard tests of IDH mutation status and MGMT methylation status alone. In addition, while other major cancers (e.g.; breast cancer, prostate cancer, ovarian cancer, lung cancer, colorectal cancer) have FDA-cleared or -approved CDx assays for specific therapies, no approved COx assays are commercially available for treatment of GBM or any form of brain cancer.

The present invention features in vitro methods for determining prognosis of a patient who has GBM or a condition that causes or is caused by dysfunction of the Wnt or insulin-like growth factor signaling pathway. In preferred embodiments, the method comprises first measuring expression levels of a panel of six (6) genes (e.g., WIF1, NGFR, IGFBP3, IBSP, HIST1H3G, and COL1A2) and secondly evaluating an isocitrate dehydrogenase 1 (IDH) mutation status and the MGMT promoter methylation status in a biological sample from the patient with the condition. Non-limiting examples of said biological sample comprise FFPE or frozen tumor tissue, possibly cerebrospinal fluid, and/or blood (e.g. circulating tumor DNA). A prognostic index is then determined from the expression of the above mentioned genes. The prognostic index comprises age of the patient at time of diagnosis or at initial presentation of the condition, the measured expression level of WIF1, NGFR, IGFBP3, IBSP, HIST1H3G, and COL1A2 as well as IDH mutation status and MGMT methylation status. This 9-factor prognostic index predicts OS of the patient with high probability (as shown in FIGS. 2A-2B). The prognosis of the patient is then based on this predicted OS of the patient. In other embodiments, the method further comprises determining prognosis of said patient based on said differential overall survival prediction. This same approach can be used for monitoring the condition longitudinally over time. Non-limiting examples of the monitoring periods comprise: 1) at time of diagnosis or initial presentation of said condition (e.g., GBM); 2) 5-7 days post-diagnosis; 3) one-month post-diagnosis, 3) three months post-diagnosis, or 4)>three months post-diagnosis. In preferred embodiments, the differential expression of WIF1 causes dysfunction of the Wnt signaling pathway. A non-limiting example that may cause Wnt signaling dysfunction comprises below average expression of WIF1, an inhibitory factor of Wnt signaling, resulting in increased Wnt signaling, which has been proposed to play a role in cancer.

The present invention further features methods for 1) treating a patient and 2) monitoring over time the patient being treated, wherein the patient has a condition that causes or is caused by dysfunction of the Wnt signaling pathway (e.g., GBM) and is in need of such treatment and treatment monitoring. In preferred embodiments, the method comprises first measuring expression levels of a panel six (6) genes (e.g., WIF1, NGFR, IGFBP3, IBSP, HIST1H3G, and COL1A2) and secondly evaluating an isocitrate dehydrogenase 1 (IDH) mutation status and the MGMT promoter methylation status in a biological sample from the patient with the condition. Non-limiting examples of said biological sample comprise tissue, cerebrospinal fluid, and/or blood. A prognostic index is then determined. The prognostic index comprises age of said patient at time of diagnosis or at initial presentation of the condition and the measured expression level of WIF1, NGFR, IGFBP3, IBSP, HIST1H3G, and COL1A2 as well as IDH mutation status and MGMT methylation status. This 9-factor prognostic index predicts OS of the patient with high probability (as shown in FIGS. 2A-2B). In preferred embodiments, differential expression of WIF1 causes dysfunction of the Wnt signaling pathway. A non-limiting example of differential expression that causes dysfunction of Wnt signaling comprises below average expression level of WIF1 combined with above average expression level of IGFBP3. A therapeutically effective drug or intervention is then administered to the patient to treat the condition based on the prognostic index. Non-limiting examples of the drug or intervention comprise standard of care agents (e.g., anti-cancer agents, radiation) and/or agents that specifically interact with or modulate Wnt signaling (e.g., an inhibitor to the Wnt signaling pathway). The patient being treated for the condition also can be monitored longitudinally over time using the prognostic index. Non-limiting examples of the monitoring periods comprise: 1) at time of diagnosis or initial presentation of said condition (e.g., GBM); 2) 5-7 days post-diagnosis; 3) one-month post-diagnosis, 3) three months post-diagnosis, or 4)>three months post-diagnosis.

The present invention further features in vitro methods for 1) determining a risk of recurrence or progression (e.g., prognosis) of a patient with GBM and 2) monitoring progression of GBM. In preferred embodiments, the method comprises first measuring expression levels of a panel of six (6) genes (e.g., WIF1, NGFR, IGFBP3, IBSP, HIST1H3G, and COL1A2) and secondly evaluating an isocitrate dehydrogenase 1 (IDH) mutation status and the MGMT promoter methylation status in a biological sample from the patient with the condition. Non-limiting examples of said biological sample comprise tissue, cerebrospinal fluid, and/or blood. A GPI (GBM prognostic index) is then determined. The GPI comprises age of the patient at time of diagnosis or at initial presentation of GBM, the measured expression level of WIF1, NGFR, IGFBP3, IBSP, HIST1H3G, and COL1A2 as well as IDH mutation status and MGMT methylation status. This 9-factor prognostic: index predicts OS of the patient with high probability (as shown in FIGS. 2A-2B). The risk of recurrence or progression of GBM can then be determined from the OS prediction. This same approach can be used for monitoring GBM longitudinally over time (e.g. at time of diagnosis or initial presentation of GBM; 5-7 days post-diagnosis; one-month post-diagnosis, three months post-diagnosis, or >three months post-diagnosis. In some embodiments, differential expression WIF1 causes dysfunction of the Wnt signaling pathway. A non-limiting example that causes dysfunction of Wnt signaling may comprise below average expression (e.g., under expression or low expression) of WIF1. In preferred embodiments, WIF1 expression is assessed in combination with IGFBP3 expression, where below average expression of WIF1 and/or above average expression (e.g., over-expression or high expression) of IGFBP3 predicts differential survival of patients with GBM.

Additionally, the present invention features a method comprising obtaining biological samples from a patient and determining a prognostic index. In some embodiments, the prognostic index is determined by measuring expression level of one or more of the following biomarkers WIF1, IGFBP3, NGFR, IBSP, HISTLH3G, and COL1A2 and evaluating the IDH mutation status and the MGMT promoter methylation status in a biological sample from said patient. Non-limiting examples of said biological sample comprise tissue, cerebrospinal fluid, and/or blood. The prognostic index comprises age of said patient at time of diagnosis or at initial presentation of the condition and the measured expression level of WIF1, NGFR, IGFBP3, IBSP, HIST1H3G, and COL1A2 as well as IDH mutation status and MGMT methylation status. This 9-factor prognostic: index predicts OS of the patient with high probability (as shown in FIGS. 2A-2B). In some embodiments, the prognostic index predicts differential overall survival of said patient. In some embodiments, the prognostic index is analysed to determine the patient's prognosis.

As previously mentioned, there remains a need for an individualized/personalized treatment of GBM based on identifying specific biomarkers and gene expression profiles (e.g., disease profiles). The present invention addresses the industry need as classification of disease profiles allow physicians to conduct individualized targeted treatment, thus improving clinical outcomes for patients with GBM.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The patent application or application file contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIGS. 1A and 1B show a heatmap of pathway scores between samples of GBM and non-neoplastic brain tissue (FIG. 1A) and between the GBM tissues with Long OS versus those with Short OS (FIG. 1B). Samples are in rows while pathways are shown in columns and the warmer the color the more upregulated the pathway is while the cooler the color the more downregulated the pathway is in that comparison.

FIGS. 1C and 1D show the average pathway scores for each group for each comparison.

FIGS. 2A, 2B, 2C, 2D, 2E, 2F, 2G, 2H, 2I, 2J, 2K, 2L, 2M and 2N show Kaplan-Meier plots. FIGS. 2A and 2B show Kaplan-Meier OS probability estimates comparing patients grouped by GBM prognostic index (GPI) within an original institutional cohort of 24 GBM patients (FIG. 2A) or within the 239 TCGA patients (FIG. 2B). FIGS. 2C, 2D, 2E, 2F, 2G, 2H, 2I, 2J, 2K, 2L, 2M and 2N show Kaplan-Meier plots of OS of patients grouped by High or Low expression of each individual gene within the GPI in both cohorts.

FIGS. 3A, 3B, 3C, 3D, 3E, and 3F show the correlation between age and expression levels in both cohorts. Scatter plots showing distribution of log 2 transformed gene expression values on the y-axis with the patient's age on the X-axis for both The Cancer Genome Atlas (TCGA) cohort (dots) and the present study's cohort (triangles) for the 6 genes included in the GPI. Correlation coefficients and associated p-values are shown in text within each figure.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

As used herein, “administering” and the like refer to the act physically delivering a composition or other therapy (e.g. a radiation therapy) described herein into a subject by such routes as oral, mucosal, topical, transdermal, suppository, intravenous, parenteral, intraperitoneal, intramuscular, intralesional, intrathecal, intranasal or subcutaneous administration. Parenteral administration includes intravenous, intramuscular, intra-arterial, intradermal, subcutaneous, intraperitoneal, intraventricular, and intracranial administration. Radiation therapy can be administered using techniques described herein, including for example, external beam radiation or brachytherapy. When a disease, disorder or condition, or a symptom thereof, is being treated, administration of the substance typically occurs after the onset of disease, disorder or condition or symptoms thereof. When a disease, disorder or condition, or symptoms thereof, are being prevented, administration of the substance typically occurs before the onset of the disease, disorder or condition or symptoms thereof.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder or condition described herein. In another embodiment, the subject is a mammal (e.g., a human) at risk of developing a disease, disorder or condition described herein. In certain instances, the term patient refers to a human.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “cancer” refers to any physiological condition in mammals characterized by unregulated cell growth. Cancers described herein include solid tumors and hematological (blood) cancers. A “hematological cancer” refers to any blood borne cancer and includes, for example, myelomas, lymphomas and leukemias. A “solid tumor” or “tumor” refers to a lesion and neoplastic cell growth and proliferation, whether malignant or benign, and all precancerous and cancerous cells and tissues resulting in abnormal tissue growth. “Neoplastic,” as used herein, refers to any form of dysregulated or unregulated cell growth, whether malignant or benign, resulting in abnormal tissue growth.

A refractory, resistant, or persistent cancer refers to a circumstance where patients, even after intensive treatment, have residual cancer cells (e.g., leukemia cells, lymphoma cells, circulating tumor cells or cancer stem cells) in their lymphatic system, blood and/or blood forming tissues (e.g., marrow).

The terms “manage,” “managing,” and “management” refer to preventing or slowing the progression, spread or worsening of a disease or disorder, or of one or more symptoms thereof. In certain cases, the beneficial effects that a subject derives from a prophylactic or therapeutic agent do not result in a cure of the disease or disorder.

The term “effective amount” as used herein refers to the amount of a therapy (e.g., an anti-cancer agent or radiation therapy provided herein) which is sufficient to reduce and/or ameliorate the severity and/or duration of a given disease, disorder or condition and/or a symptom related thereto. This term also encompasses an amount necessary for the reduction or amelioration of the advancement or progression of a given disease (e.g., cancer), disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” of an anti-cancer agent or a radiation therapy described herein is an amount sufficient to provide a therapeutic benefit in the treatment or management of a cancer, or to delay or minimize one or more symptoms associated with the presence of the cancer. A therapeutically effective amount of an anti-cancer agent described herein, or a radiation therapy described herein means an amount of therapeutic agent, alone or in combination with other therapies, which provides a therapeutic benefit in the treatment or management of the cancer. The term “therapeutically effective amount” can encompass an amount that improves overall therapy, reduces or avoids symptoms or causes of cancer, or enhances the therapeutic efficacy of another therapeutic agent.

A therapy is any protocol, method and/or agent that can be used in the prevention, management, treatment and/or amelioration of a given disease, disorder or condition. In certain embodiments, the terms “therapies” and “therapy” refer to a drug therapy, biological therapy, supportive therapy, radiation therapy, and/or other therapies useful in the prevention, management, treatment and/or amelioration of a given disease, disorder or condition known to one of skill in the art such as medical personnel.

A regimen is a protocol for dosing and timing the administration of one or more therapies (e.g., combinations described herein, another active agent such as for example an anti-cancer agent described herein, or a radiation therapy described herein) for treating a disease, disorder, or condition described herein. A regimen can include periods of active administration and periods of rest as known in the art. Active administration periods include administration of combinations and compositions described herein and the duration of time of efficacy of such combinations, compositions, and radiation therapies. Rest periods of regimens described herein include a period of time in which no agent (e.g., a polypeptide described herein or an anti-cancer agent described herein) is actively administered, and in certain instances, includes time periods where the efficacy of such agents can be minimal. Rest periods of regimens described herein can include a period of time in which no radiation therapy is actively administered. Combination of active administration and rest in regimens described herein can increase the efficacy and/or duration of administration of the combinations described herein.

The term “anti-cancer agent” is used in accordance with its plain ordinary meaning and refers to a composition having anti-neoplastic properties or the ability to inhibit the growth or proliferation of cells. In certain embodiments, an anti-cancer agent is chemotherapeutic. In certain embodiments, an anti-cancer agent is an agent identified herein having utility in methods of treating cancer. In certain embodiments, an anti-cancer agent is an agent approved by the FDA or similar regulatory agency of a country other than the USA, for treating cancer.

The term “chemotherapeutic” or “chemotherapeutic agent” is used in accordance with its plain ordinary meaning and refers to a chemical composition or compound having anti-neoplastic properties or the ability to inhibit the growth or proliferation of cells. “Chemotherapy” or “cancer therapy” refers to a therapy or regimen that includes administration of a combination, chemotherapeutic, or anti-cancer agent described herein.

The term “radiation therapy” is used in accordance with its plain ordinary meaning and refers to the medical use of radiation in the treatment of cancer. Preferably, the medical use of radiation in the treatment of cancer results in the killing of cancer cells in the subject. A variety of radiation therapies as anti-cancer agents can be used in accordance with the present disclosure, examples of which are provided herein.

Companion Diagnostic (CDx) assays, as defined by the FDA, are in vitro diagnostics (IVD) devices that provide information essential for the safe and effective use of a corresponding therapeutic product. The FDA specifies three main areas where a CDx assay is essential: 1) Identify patients who are most likely to benefit from a particular therapeutic product; 2) Identify patients likely to be at increased risk of serious adverse reactions as a result of treatment with a particular therapeutic product; and 3) To monitor response to treatment for the purpose of adjusting treatment (e.g., schedule, dose, discontinuation) and to achieve improved safety or effectiveness. A CDx can be used both to predict outcome (efficacy and safety) and to monitor response. The FDA has approved or cleared 35 CDx devices (as of 2018), which are available for the treatment of specific leukemias, gastrointestinal tumors, breast cancers, ovarian cancers, melanomas, lung cancers, and colorectal cancers, while no approved CDx assays are available for the treatment of GBM or any form of brain cancer.

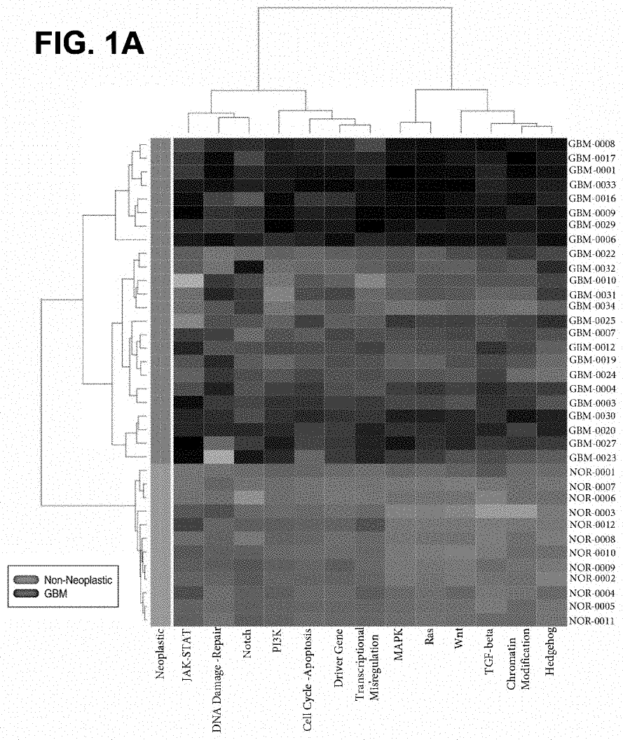

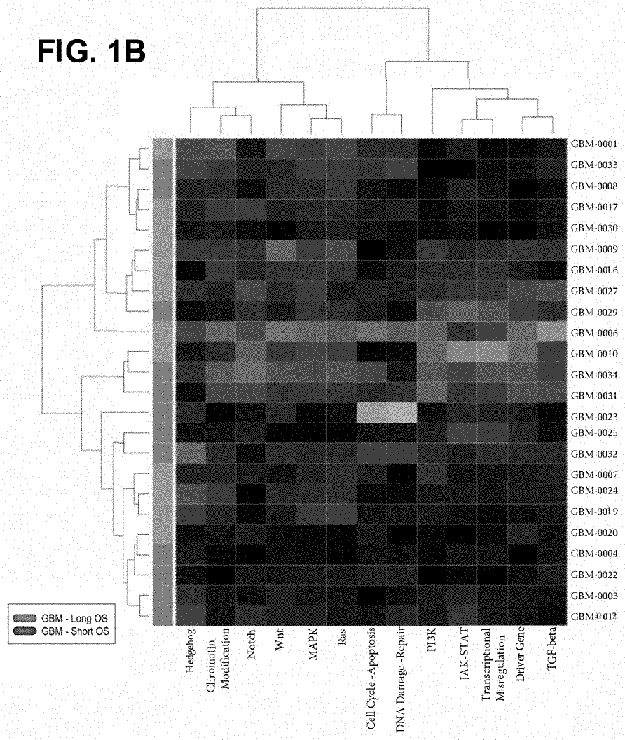

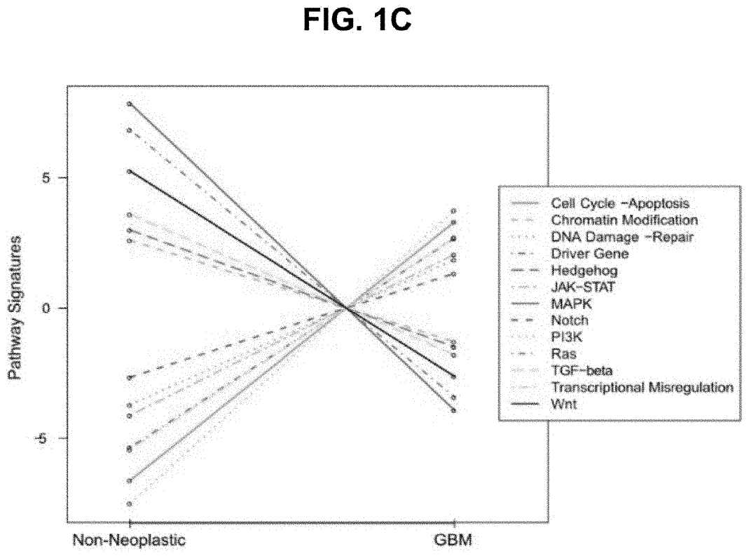

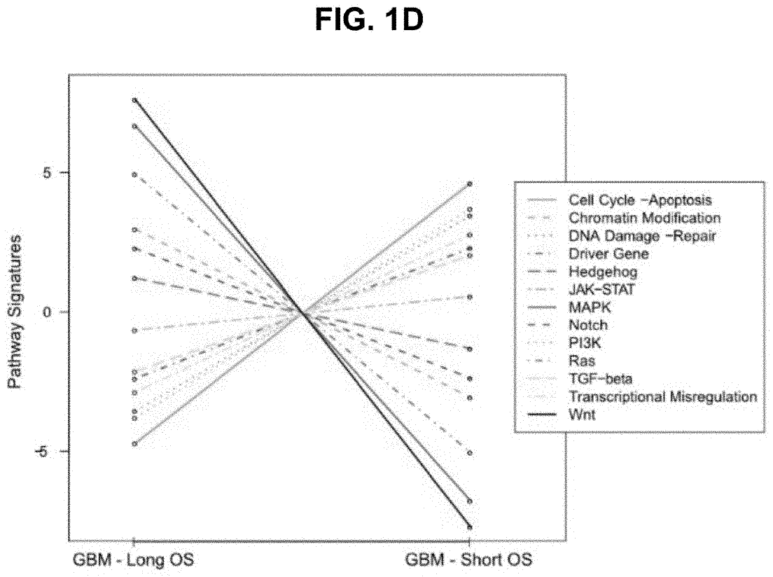

Referring now to FIGS. 1A-3F, the present invention features a new method for developing personalized treatment strategies for GBM and for conditions that cause or are caused by dysfunctional Wnt or insulin-like growth factor signaling using the expression and/or activity of WIF1 and IGFBP3 for prognostic purposes. This technology allows for a method to use gene expression profiles to classify patients into prognostic groups which could tailor treatment and monitoring strategies for GBM or disease conditions that are caused by similar signaling dysregulation. In some embodiments, the present invention uses changes in select signaling pathways (e.g., Wnt signaling, IGF signaling, NGF signaling) as indicators for GBM prognosis, disease progression, and personalized treatment and monitoring strategies.

Non-limiting examples of the advantages of this technology comprise: 1) personalized medicine strategies for optimal individual treatment of patients with GBM; 2) ability to stratify patients with GBM into prognostic groups 3) preventing progression of GBM; and 4) treating GBM.

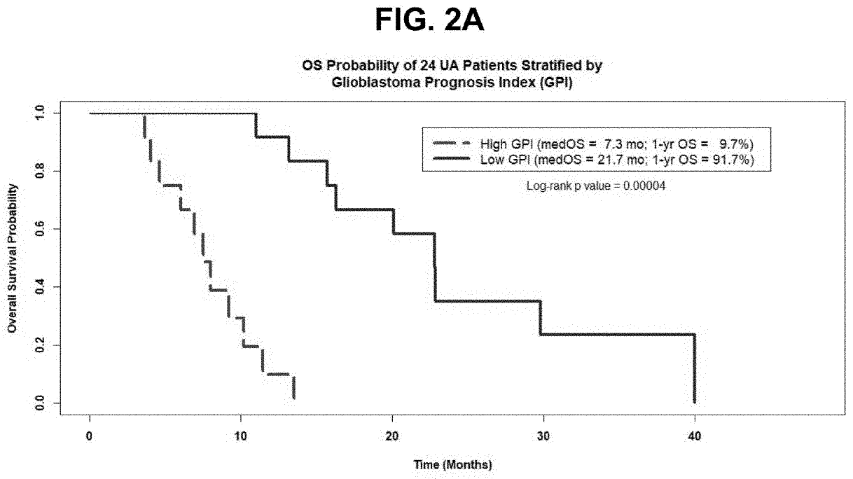

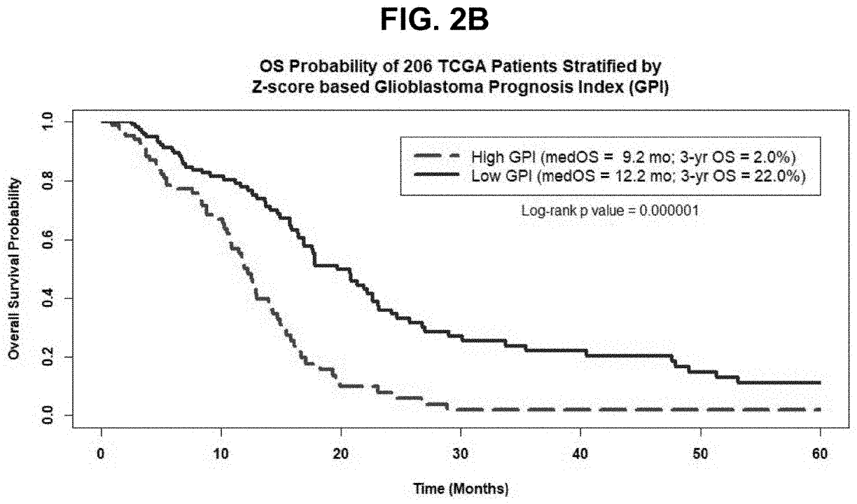

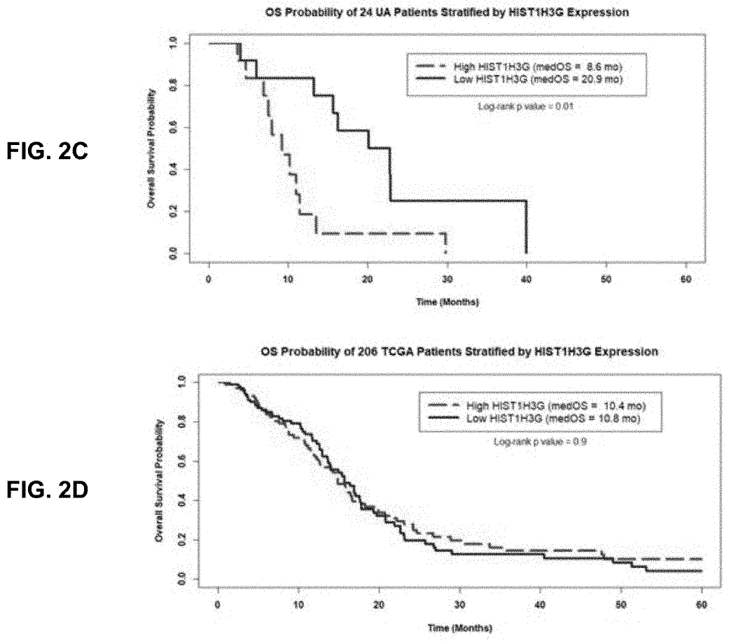

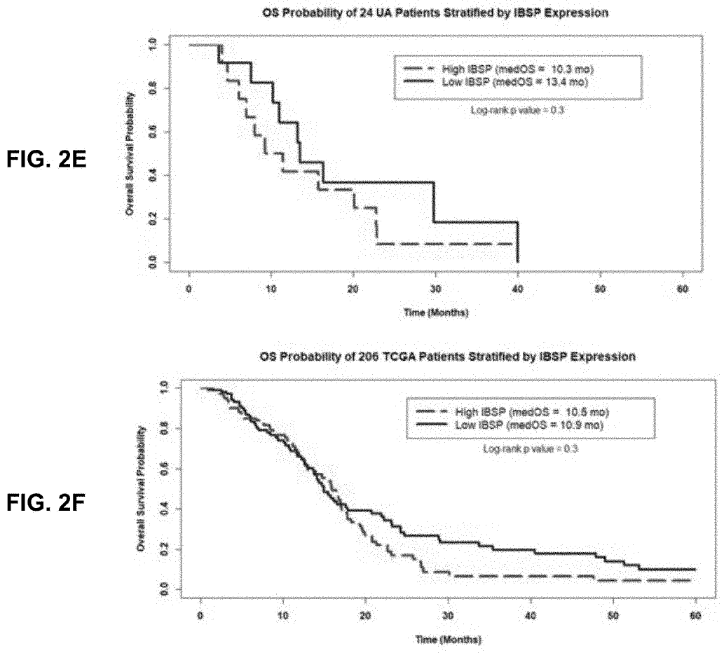

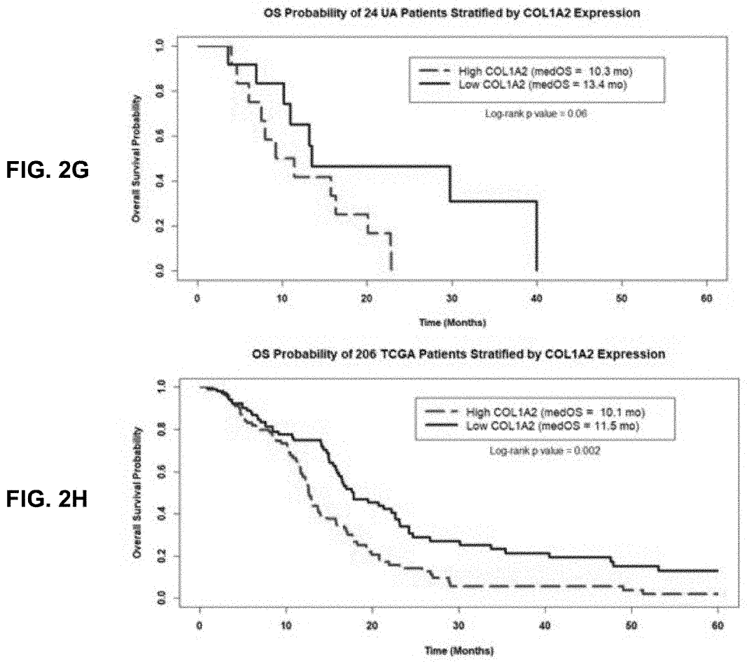

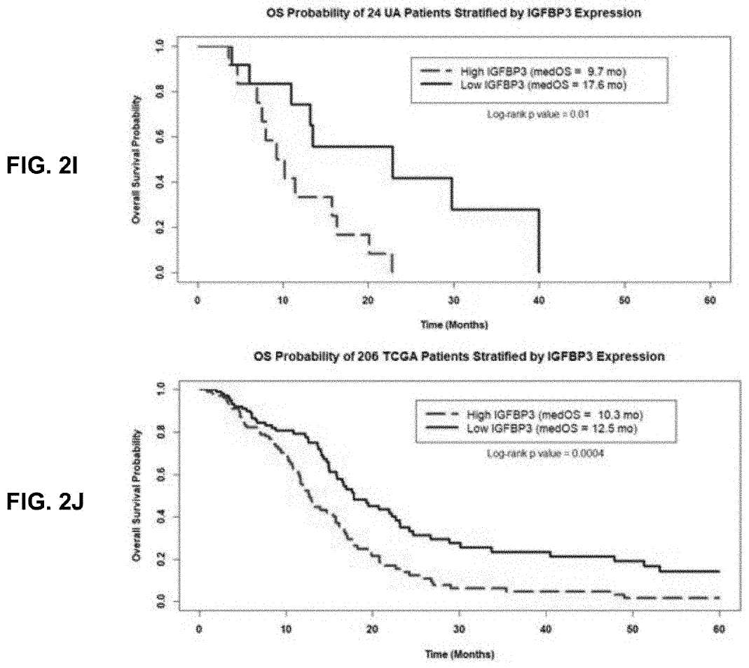

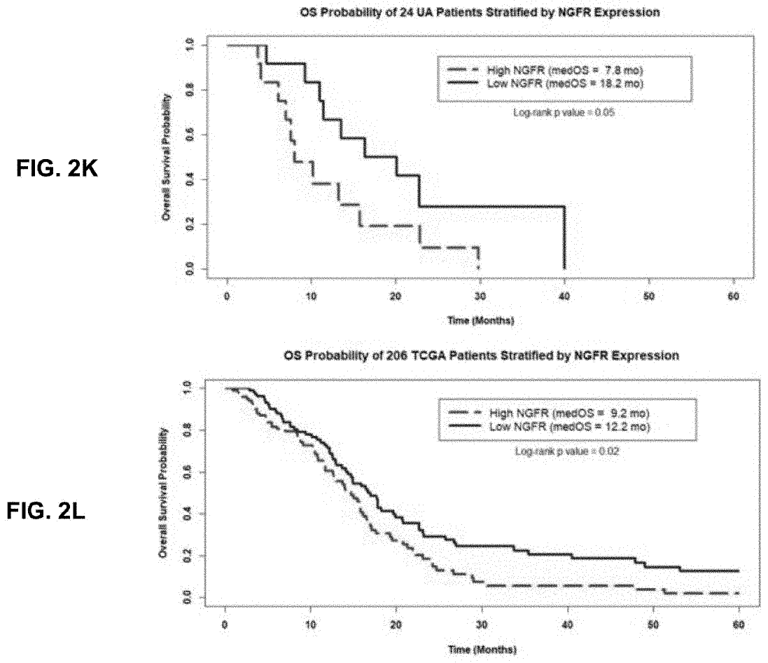

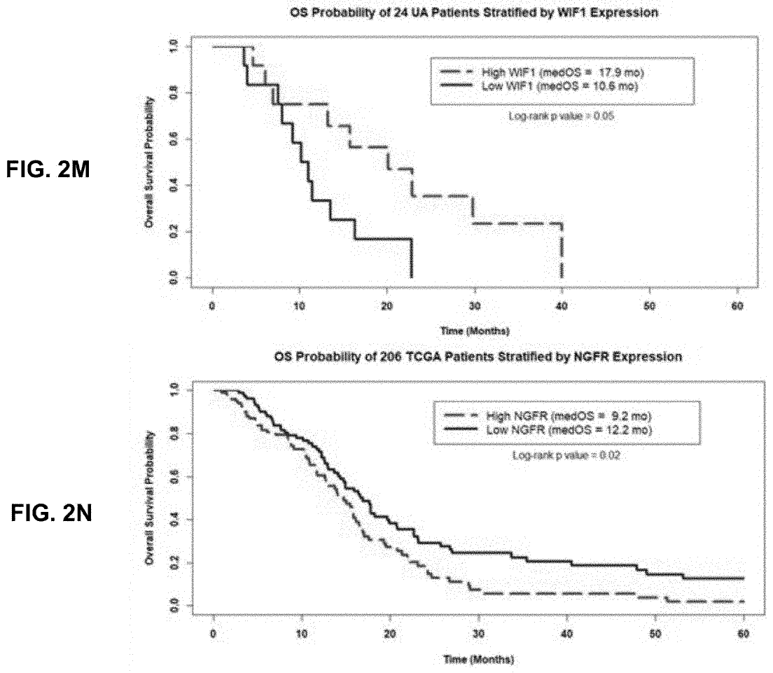

The present invention features in vitro methods for determining prognosis of a patient who has GBM or a condition that causes or is caused by dysfunction of the Wnt or insulin-like growth factor signaling pathway. In preferred embodiments, the method comprises first measuring expression levels of a panel of six (6) genes (e.g., WIF1, NGFR, IGFBP3, IBSP, HIST1H3G, and COL1A2) and secondly evaluating an isocitrate dehydrogenase 1 (IDH) mutation status and the MGMT (O6-Methylguanine-DNA Methyltransferase) promoter methylation status in a biological sample from the patient with the condition. Non-limiting examples of said biological sample comprise Formalin-Fixed Paraffin-Embedded (FFPE) tissue or frozen tumor tissue, possibly cerebrospinal fluid, and/or blood (e.g. circulating tumor DNA). A prognostic index is then determined from the expression of these genes as well as the IDH mutation status and MGMT promoter methylation status. The prognostic index comprises age of the patient at time of diagnosis or at initial presentation of the condition, the measured expression level of one or more of the aforementioned biomarkers (WIF1, NGFR, IGFBP3, IBSP, HIST1H3G, and COL1A2) as well as IDH mutation status and MGMT methylation status. This 9-factor prognostic index predicts OS of the patient with high probability (as shown in FIGS. 2A-2B). The prognosis of the patient is then based on this predicted OS of the patient. This same approach can be used for monitoring the condition longitudinally over time. Non-limiting examples of the monitoring periods comprise: 1) at time of diagnosis or initial presentation of said condition (e.g., GBM); 2) 5-7 days post-diagnosis; 3) one-month post-diagnosis, 3) three months post-diagnosis, or 4)>three months post-diagnosis. In preferred embodiments, the differential expression of WIF1 causes dysfunction of the Wnt signaling pathway. A non-limiting example that may cause Wnt signaling dysfunction comprises below average expression of WIF1, an inhibitory factor of Wnt signaling, resulting in increased Wnt signaling, which has been proposed to play a role in cancer.

The present invention also features an in vitro method far monitoring disease progression of a patient who has a condition that causes or is caused by dysfunction of Wnt signaling pathway. In some embodiments, the method comprises first measuring expression levels of panel of six (6) genes (e.g., WIF1, NGFR, IGFBP3, IBSP, HIST1H3G, and COL1A2) and secondly evaluating an IDH mutation status and the MGMT promoter methylation status in a biological sample from the patient with the condition. A prognostic index is then determined from the expression of the aforementioned genes as well as the IDH mutation status and MGMT promoter methylation status. The prognostic index comprises age of the patient at time of diagnosis or at initial presentation of the condition, the measured expression level of one or more of the aforementioned biomarkers (WIF1, NGFR, IGFBP3, IBSP, HIST1H3G, and COL1A2) as well as IDH mutation status and MGMT methylation status. This 9-factor prognostic index predicts OS of the patient with high probability (as shown in FIGS. 2A-2B). In further embodiments, the method comprises monitoring the disease progression of said patient based on said overall survival prediction. Non-limiting conditions that causes or are caused by, in part, dysfunction of Wnt signaling include but are not limited to Glioblastoma Multiforme (GBM), cancers that are radio-resistant or eligible to receive radiation therapy, or any condition that causes or is caused by Wnt signaling or insulin-like growth factor signalling dysfunction, or the condition comprises in part dysregulation in signaling pathways comprising NGFR, IBSP, and/or TP53.

In some embodiments, the present invention features an in vitro method for determining a risk of recurrence or progression of Glioblastoma Multiforme (GBM) for a patient suffering from said GBM. In some embodiments, the method comprises first measuring expression levels of a panel of six (6) genes (e.g., WIF1, NGFR, IGFBP3, IBSP, HIST1H3G, and COL1A2) and secondly evaluating an IDH mutation status and the MGMT promoter methylation status in a biological sample from the patient with the condition. A GBM prognostic index (GPI) is then determined from the expression of at least one of the aforementioned genes as well as the IDH mutation status and MGMT promoter methylation status. The prognostic index comprises age of the patient at time of diagnosis or at initial presentation of the condition, the measured expression level of one or more of the aforementioned biomarkers (WIF1, NGFR, IGFBP3, IBSP, HIST1H3G, and COL1A2) as well as IDH mutation status and MGMT methylation status. This 9-factor prognostic index predicts OS of the patient with high probability (as shown in FIGS. 2A-2B). In further embodiments, the method comprises determining risk of recurrence or progression of said GBM based on said differential overall survival.

The present invention further features methods for 1) treating a patient and 2) for monitoring treatment of a patient over time the patient being treated, wherein the patient has a condition that causes or is caused by dysfunction of the Wnt signaling pathway (e.g., GBM) and is in need of such treatment and treatment monitoring. In preferred embodiments, the method comprises first measuring expression levels of a panel six (6) genes (e.g., WIF1, NGFR, IGFBP3, IBSP, HIST1H3G, and COL1A2) and secondly evaluating the mutation status of IDH and the MGMT promoter methylation status in a biological sample from the patient with the condition. Non-limiting examples of said biological sample comprise tissue, cerebrospinal fluid, and/or blood. A prognostic index (or GBM prognostic index, GPI) is then determined. The prognostic index comprises age of said patient at time of diagnosis or at initial presentation of the condition and the measured expression level of at least one of the aforementioned genes (WIF1, NGFR, IGFBP3, IBSP, HIST1H3G, and COL1A2) as well as IDH mutation status and MGMT methylation status. This 9-factor prognostic index predicts OS of the patient with high probability (as shown in FIGS. 2A-2B). In further embodiments, the method comprises administering a therapeutically effective amount of drug or intervention to said patient to treat said condition based on said prognostic index or the method may comprise monitoring over time said patient being treated for said condition based on said prognostic index. In preferred embodiments, differential expression of WIF1 causes dysfunction of the Wnt signaling pathway. A non-limiting example of differential expression that causes dysfunction of Wnt signaling comprises below average expression level of WIF1 combined with above average expression level of IGFBP3. A therapeutically effective drug or intervention is then administered to the patient to treat the condition based on the prognostic index. Non-limiting examples of the drug or intervention comprise standard of care agents (e.g., anti-cancer agents, radiation) and/or agents that specifically interact with or modulate Wnt signaling (e.g., an inhibitor to the Wnt signaling pathway). The patient being treated for the condition also can be monitored longitudinally over time using the prognostic index. Non-limiting examples of the monitoring periods comprise: 1) at time of diagnosis or initial presentation of said condition (e.g., GBM); 2) 5-7 days post-diagnosis; 3) one-month post-diagnosis, 3) three months post-diagnosis, or 4)>three months post-diagnosis.

The present invention further features in vitro methods for 1) determining a risk of recurrence or progression (e.g., prognosis) of a patient with GBM and 2) monitoring progression of GBM. In preferred embodiments, the method comprises first measuring expression levels of a panel of six (6) genes (e.g., WIF1, NGFR, IGFBP3, IBSP, HIST1H3G, and COL1A2 and secondly evaluating the mutation status of IDH and the MGMT promoter methylation status in a biological sample from the patient with the condition. Non-limiting examples of said biological sample comprise tissue, cerebrospinal fluid, and/or blood. A GPI is then determined. The GPI comprises age of the patient at time of diagnosis or at initial presentation of GBM, the measured expression level of WIF1, NGFR, IGFBP3, IBSP, HIST1H3G, and COL1A2 as well as IDH mutation status and MGMT methylation status. This 9-factor prognostic index predicts OS of the patient with high probability (as shown in FIGS. 2A-2B). The risk of recurrence or progression of GBM can then be determined from the OS prediction. This same approach can be used for monitoring GBM longitudinally over time (e.g. at time of diagnosis or initial presentation of GBM; 5-7 days post-diagnosis; one-month post-diagnosis, three months post-diagnosis, or >three months post-diagnosis. In some embodiments, differential expression WIF1 causes dysfunction of the Wnt signaling pathway. A non-limiting example that causes dysfunction of Wnt signaling may comprise below average expression (e.g., under expression or low expression) of WIF1. In preferred embodiments, WIF1 expression is assessed in combination with IGFBP3 expression, where below average expression of WIF1 and/or above average expression (e.g., over-expression or high expression) of IGFBP3 predicts differential survival of patients with GBM.

The present invention features a method for treating Glioblastoma Multiforme (GBM) in a patient in need thereof. In some embodiments, the method comprises determining a GBM prognostic index (GPI). In some embodiments GPI is determined by measuring expression level of one or more of the following biomarkers WIF1, IGFBP3, NGFR, IBSP, HISTLH3G, and COL1A2 and evaluating an isocitrate dehydrogenase 1 (IDH) mutation status and the MGMT (O6-Methylguanine-DNA Methyltransferase) promoter methylation status in a biological sample from said patient. In some embodiments, the GPI comprises age of said patient, said measured expression level of one or more WIF1, IGFBP3, NGFR, IBSP, TP53, HISTLH3G, and COL1A2, and said IDH mutation status and MGMT promoter methylation status. In some embodiments, the GPI predicts differential overall survival of said patient. In some embodiments, the method comprises administering a therapeutically effective amount of drug or intervention to said patient to treat GBM based on said prognostic index.

The present invention may further feature a method of stratifying patients with Glioblastoma Multiforme (GBM). In some embodiments, the method comprises determining a GBM prognostic index (GPI). In some embodiments GPI is determined by measuring expression level of one or more of the following biomarkers WIF1, IGFBP3, NGFR, IBSP, HISTLH3G, and COL1A2 and evaluating an isocitrate dehydrogenase 1 (IDH) mutation status and the MGMT (O6-Methylguanine-DNA Methyltransferase) promoter methylation status in a biological sample from said patient. In some embodiments, the GPI comprises age of said patient, said measured expression level of one or more WIF1, IGFBP3, NGFR, IBSP, TP53, HISTLH3G, and COL1A2, and said IDH mutation status and MGMT promoter methylation status. In some embodiments, the GPI predicts differential overall survival (OS) of said patient. In some embodiments, patients with a longer OS relative to median OS are determined to have a good prognosis. In other embodiments, patients with a shorter OS relative to median OS are determined to have a poor prognosis.

The present invention also features a method for treating a patient in need thereof who has a condition that causes or is caused by dysfunction of the Wnt signaling pathway. In some embodiments said method comprises determining the prognostic index of the patient. In some embodiments, the prognostic index is determined by measuring expression level of one or more of the following biomarkers WIF1, IGFBP3, NGFR, IBSP, HISTLH3G, and COL1A2; and evaluating an isocitrate dehydrogenase 1 (IDH) mutation status and the MGMT (O6-Methylguanine-DNA Methyltransferase) promoter methylation status in a biological sample from said patient. In some embodiments, the prognostic index comprises the age of said patient, said measured expression level of one or more WIF1, IGFBP3, NGFR, IBSP, TP53, HISTLH3G, and COL1A2, and said IDH mutation status and MGMT promoter methylation status. In some embodiments, the prognostic index predicts differential overall survival of said patient. In some embodiments, the method comprises administering a therapeutically effective amount of drug or intervention to said patient to treat said condition based on said prognostic index.

The present invention features a method of monitoring effectiveness of a treatment that is currently being administered to a patient in need thereof who has a condition that causes or is caused by dysfunction of the Wnt signaling pathway. In some embodiments, the method comprises obtaining a biological sample from the patient. In other embodiments, the method comprises determining the prognostic index of the patient. In some embodiments, determining a prognostic index comprises measuring expression level of one or more of the following biomarkers WIF1, IGFBP3, NGFR, IBSP, HISTLH3G, and COL1A2 in the biological sample from said patient and evaluating an isocitrate dehydrogenase 1 (IDH) mutation status and the MGMT (O6-Methylguanine-DNA Methyltransferase) promoter methylation status. In some embodiments, the prognostic index comprises the age of said patient, said measured expression level of one or more WIF1, IGFBP3, NGFR, IBSP, TP53, HISTLH3G, and COL1A2, said IDH mutation status and MGMT promoter methylation status. In some embodiments, the prognostic index predicts differential overall survival of said patient. In some embodiments, the method comprises administering a different treatment to the patient if the patient has a poor prognosis. In some embodiments, a poor prognosis is determined when the patient has a shorter overall survival compared to a median overall survival. In other embodiments, the method comprises maintaining the current treatment of the patient, if the patient has a good prognosis. In some embodiments, a good prognosis is determined when the patient has a longer overall survival compared to a median overall survival.

In other embodiments, the treatment administered to the patient is the same treatment, at a higher lower dose. In some embodiments, the treatment administered to the patient is a different treatment. In other embodiments, the different treatment administered to the patient is the same treatment, at a higher dose. In some embodiments, the patient is administered the same treatment in combination with another treatment. In other embodiments, the treatment administered to the patient is the same treatment, at a lower dose.

The present invention may further feature a method of stratifying patients who have a condition that causes or is caused by dysfunction of the Wnt signaling pathway. In some embodiments, the method comprises determining a prognostic index. In some embodiments the prognostic index is determined by measuring expression level of one or more of the following biomarkers WIF1, IGFBP3, NGFR, IBSP, HISTLH3G, and COL1A2 and evaluating an isocitrate dehydrogenase 1 (IDH) mutation status and the MGMT (O6-Methylguanine-DNA Methyltransferase) promoter methylation status in a biological sample from said patient. In some embodiments, the prognostic index comprises age of said patient, said measured expression level of one or more WIF1, IGFBP3, NGFR, IBSP, TP53, HISTLH3G, and COL1A2, and said IDH mutation status and MGMT promoter methylation status. In some embodiments, the prognostic index predicts differential overall survival (OS) of said patient. In some embodiments, patients with a longer OS relative to median OS are determined to have a good prognosis. In other embodiments, patients with a shorter OS relative to median OS are determined to have a poor prognosis.

In some embodiments, the biological sample obtained from said patient comprises tissue, cerebrospinal fluid, and/or blood.

In appropriate circumstances, the condition comprises glioblastoma multiforme (GBM) or a condition that may in part be caused by Wnt signaling dysfunction comprising GBM, any other cancers that are radiation-resistant or eligible to receive radiation treatment, such as breast, lung, head and neck, pancreatic, or prostate cancer, or any condition or treatment that causes or is caused by Wnt signaling or insulin-like growth factor signalling dysfunction. In other circumstances, the condition also may comprise in part conditions with similar dysregulation in the other identified pathways involving NGF, IBSP, and/or TP53.

In preferred embodiments, the markers of the prognostic index (e.g., GPI) comprise WIF1, NGFR, IGFBP3, IBSP, HIST1H3G, and COL1A2 or a combination thereof. In other embodiments, markers may comprise members of other signaling pathways comprising FZD9, WNT7B, SFRP1, FZD7, PRKCG, PRKCB, PPP3CB, CCND1, TP53, COL4A6, MMP9, MMP7 or a combination thereof. In appropriate circumstances, WIF1 (e.g., protein, mRNA, or gene) can be used as a radiosensitizer, an agent that makes tumor cells more sensitive to radiation or chemotherapy therapy.

In some embodiments, expression comprises the protein expression, gene expression (e.g., mRNA levels, DNA levels, and/or protein activity level) of signaling factors, of WIF1, NGFR, IGFBP3, IBSP, HIST1H3G, and COL1A2 or other select Wnt or insulin-like growth factor pathway family members. In other embodiments, measuring signaling comprises measuring the protein expression level, gene expression level (or mRNA levels), DNA levels, and/or protein activity of signaling factors. In some embodiments, measuring Wnt signaling pathway dysfunction is based on disease profiles identified through differential expression (of protein levels, mRNA levels, DNA levels, and/or activity) of Wnt signaling pathway family members. Differential expression can be measured through standard and emerging proteomic and genomic technologies, including but not limited to immunohistochemistry, fluorescence in situ hybridization, protein arrays, mass spectroscopy, gene arrays, ELISA, RT-PCR, PCR, and next generation sequencing.

In some embodiments, the expression level of WIF1 causes dysfunction of the Wnt signaling pathway, wherein said dysfunction predicts overall survival of patients with GBM or said condition that causes or caused by Wnt signaling dysfunction. In other embodiments, the Wnt signaling pathway dysfunction is further based on differential expression of WIF1.

In some embodiments, the differential expression of a Wnt signaling pathway family member comprises above average expression of WIF1 or below average expression of WIF1. Non-limiting examples may comprise that above expression of WIF1 predicts longer OS and below average expression of WIF1 predicts shorter OS of the patient relative to median OS of GBM or condition that causes or caused by Wnt signaling dysfunction.

In other embodiments, the differential expression of other signaling pathways comprises above average expression of IGFBP3 or below average expression of IGFBP3. Non-limiting examples may comprise that above expression of IGFBP3 predicts shorter OS and below average expression of IGFBP3 predicts longer OS of the patient with GBM relative to median OS of GBM. In some embodiments, the differential expression of other signaling pathways comprises above average expression of NGFR or below average expression of NGFR. Non-limiting examples may comprise that above expression of NGFR predicts shorter OS and below average expression of NGFR predicts longer OS of the patient with GBM relative to median OS of GBM.

In preferred embodiments, the differential expression of genes comprises a combined expression of WIF1 and IGFBP3. Non-limiting examples may comprise that above average expression of WIF1 combined with below average expression of IGFBP3 indicates good prognosis predicting longer OS relative to median OS and wherein below average expression of WIF1 combined with above average expression of IGFBP3 comprises poor prognosis predicting shorter OS relative to median OS of patients with GBM.

In some embodiments, the GPI (i.e. the GBM prognostic index) comprises age of patient at diagnosis of GBM and the expression level of one or more WIF1, IGFBP3, NGFR, IBSP, TP53, HISTLH3G, and COL1A2, IDH mutation status and MGMT methylation status. In other embodiments, a below median GPI or low GPI, predicts a longer overall survival relative to median overall survival. In some embodiments, an above median GPI or high GPI predicts a shorter overall survival relative to median overall survival

In preferred embodiments, the markers of the prognostic index (e.g., GPI) comprise evaluating the isocitrate dehydrogenase 1 (IDH) mutation status of an individual. As used herein “IDH mutation status” may refer to whether or not the IDH protein is mutated. In some embodiments, a non-limiting example may include when IDH1 is mutated at position 132 in which an arginine is converted to a histidine (R132H). In some embodiments, IDH may be mutated at any other position. In some embodiments, a mutation in IDH may indicate a better prognosis. In some embodiments, no mutations in IDH may indicate a poorer prognosis.

In preferred embodiments, the markers of the prognostic index (e.g., GPI) comprise evaluating the MGMT (O⁶-Methylguanine-DNA Methyltransferase) promoter methylation status. In some embodiments, the CpG island of MGMT is hypermethylated within the promoter and gene itself. In some embodiments, the CpG island of MGMT is hypomethylated within the promoter and gene itself. In some embodiments, methylation(s) at the MGMT promoter may indicate a better prognosis. In other embodiments, a lack of MGMT promoter methylation may indicate poorer prognosis.

As used herein “CpG or CG sites” refer to regions of DNA where a cytosine nucleotide is followed by a guanine nucleotide in the linear sequence of bases along its 5′ 3′ direction. In some embodiments, CpG islands are located predominantly at gene promoters and other regulatory regions.

In some embodiments, the method is used as a companion diagnostic for GBM or any condition or treatment that causes or is caused by Wnt signaling dysfunction.

The present invention further features that the prognostic index or GPI may be used or measured longitudinally in cerebrospinal fluid (CSF) and/or blood (e.g., circulating tumor DNA). The disease profiles (e.g., differential expression and/or activity) of WIF1, NGFR, IGFBP3, IBSP, HIST1H3G, and COL1A2 can be measured longitudinally and longitudinal levels (e.g., profiles) can be compared to baseline levels (e.g., profiles) to detect changes in disease profiles (e.g., changes in differential expression or activity). In some embodiments, baseline measurement is performed in asymptomatic individuals, wherein asymptomatic individuals are individuals without the condition (e.g., non-neoplastic conditions). For example, blood or CSF from asymptomatic individuals or tissue from patients treated for intractable epilepsy with temporal lobe resections may be used as non-neoplastic material (e.g., non-neoplastic CSF, blood, tissue) for comparisons. In appropriate circumstances, baseline levels are normal levels from an aggregate population of asymptomatic individuals without the condition. In other circumstances, baseline levels are relative baseline levels at the time of initial diagnosis of the condition. The expression and/or activity of WIF1, NGFR, IGFBP3, IBSP, HIST1H3G, and COL1A2 also can be measured in comparator populations of individuals with a benign condition and/or different stages of the condition to identify disease profiles for various stages of the condition.

In some embodiments, the expression level of WIF1, IGFBP3, NGFR, IBSP, HISTLH3G, and COL1A2 is measured at baseline. In some embodiments, the baseline expression level is the relative baseline level at the time of initial presentation or diagnosis of the condition. In other embodiments, the baseline levels are normal levels from an aggregate population of asymptomatic individuals without the condition. In further embodiments, the expression of WIF1, IGFBP3, NGFR, IBSP, HISTLH3G, and COL1A2 are measured in comparator populations of individuals with a benign condition or at different stages of the condition.

In cerebrospinal fluid (CSF) and/or blood, WIF1, NGFR, IGFBP3, IBSP, HIST1H3G, and COL1A2 signaling dysfunction can be measured at various times, longitudinally, throughout progression of the condition. Non-limiting examples of the timing comprise: 1) at time of diagnosis; 2) 5-7 days post-diagnosis; 3) one-month post-diagnosis, 3) three months post-diagnosis, or 4)>three months post-diagnosis.

In some embodiments, the therapeutically effective drugs comprise modulators of Wnt or insulin-like growth factor signaling. Non-limiting examples include inhibitors or stimulators of these signaling pathways, which would result in altered expression or signaling activity of the genes or proteins in these pathways. In other embodiments, the therapeutically effective drugs or interventions comprise pyrvinium or XAV939 or chemical derivatives thereof, radiation, inhibitors to IGFBP3, and/or activators of WIF1. In further embodiments, therapeutically effective drug or intervention comprises temozolomide, pyrvinium or XAV939, chemical derivatives thereof, radiation, modulators of Wnt signaling, inhibitors to IGFBP3, and/or activators of WIF.

In some embodiments, the methods described herein are for personalizing initiation and continuation of therapy. In some embodiments, the methods described herein are for reducing the progression of said condition. In some embodiments, the methods described herein are for diminishing the condition or risk of said condition. In some embodiments, the methods described herein are for delaying the transition from benign to malignant disease. In some embodiments, the methods described herein are for stratifying patients with cancer or those at risk for cancer to determine appropriate medication and level of medication and/or intervention to treat progression of said cancer.

In other embodiments, the methods described herein are for longitudinal assessment of at risk and/or cancer prognosis and progression. In some embodiments, the longitudinal assessment is measured over time comprising 1) at time of diagnosis or initial presentation of said condition or GBM; 2) 5-7 days post-diagnosis; 3) one-month post-diagnosis, 3) three months post-diagnosis, or 4)>three months post-diagnosis.

In some embodiments, the methods described herein are to prolong life. In some embodiments, life is prolonged by about 1 month, or about 2 months, or about 3 months, or about 4 months, or about 5 months. In some embodiments, life is prolonged between 6-10 months. In some embodiments, life is prolonged by more than 10 months.

In some embodiments, the present invention features a method that may prolong life, for personalizing initiation and continuation of therapy, or for determining the onset of the condition (e.g., GBM). In appropriate circumstances, the method may reduce the progression of the condition (e.g., GBM) and diminish the condition (e.g., GBM) (e.g., to increase progression-free or relapse-free survival), delay the transition from benign to malignant disease as well as for a self-monitoring method and/or self-modifying of treatments. For example, using the GPI to determine risk of recurrence allows medical professionals (e.g., treating physicians) to change to a more effective or additional therapy or intervention or additional monitoring, thereby increasing time to progression and length of OS. Non-limiting examples comprise that the method may prolong life by about 1 month, 2 months, 3 months, 4 months, 5 months, 6-10 months, or >10 months.

The present invention also features a method that can be used as a companion diagnostic for any condition or treatment that causes or is caused by Wnt signaling dysfunction, for example GBM.

Other embodiments of the present invention may feature an in vitro method of stratifying patients with the condition (e.g., GBM) or those at risk for the condition (e.g., GBM) to determine appropriate medication or therapeutic intervention(s) and dosage of medication or augmentation to intervention(s) to treat further progression of the condition (e.g., GBM).

In preferred embodiments, the prognostic index score or GPI score is calculated using the normalized gene expression values of the panel of genes identified in this invention, or a panel of Z-score scaled gene expression values, and/or other statistical algorithms that combine the expression levels of these identified genes (as shown in the Examples below). A non-limiting example of a range of GPI scores comprises a range of 0-30. If the individual patient's GPI score falls in the low end of this range, in some embodiments less than 9 and in other embodiments less than 12, then that patient would be classified in the good prognosis group, while those patients with GPI scores above the cut-off would be classified as being in the poor prognosis group.

In some embodiments, the prognostic index or GPI score is calculated using the gene expression values of this panel of 6 genes, and normalized using appropriate statistical algorithms, wherein the range of prognostic or GPI scores comprises a range of 0-30 and the cut point between good and poor prognosis groups is either the median GPI score of a large cohort of patients, or determined using statistical techniques such as the maximal rank statistics test, or other appropriate statistical techniques.

In some embodiments, a “good prognosis” refers to a prognosis that indicates a longer OS relative to median OS. In other embodiments, a “poor prognosis” refers to a prognosis that indicates a shorter OS relative to median OS.

In some embodiments, a patient with a poor prognosis as predicted by the prognostic index (i.e., GPI) described herein, may be more likely to have a recurrence of cancer. In other embodiments, a patient with a poor prognosis as predicted by the prognostic index (i.e., GPI) described herein, may be more likely to have an earlier recurrence of cancer compared to a patient with a good prognosis. In some embodiments, a patient with a poor prognosis as predicted by the prognostic index (i.e., GPI) described herein, may be required to do more follow-up imaging to monitor the disease as compared to a patient with a good prognosis.

In some embodiments, a patient with a poor prognosis may be enrolled in clinical trials to test new treatment therapies of a condition described herein. In other embodiments, a patient with a good prognosis may enroll in clinical trials to test new treatment therapies of a condition described herein. In some embodiments, a patient with a poor prognosis may not receive treatment for a condition described here. In some embodiments, a patient with a poor prognosis may forgo treatment for a condition described here.

In some embodiments, the prognostic index score or GPI score is calculated using the formula GPI=0.022*Age−0.039*HIST1H3G 0.379*IBSP 0.489*COL1A2+0.046*IGFBP3+0.641*NGFR−0.039*WIF1−2.159 if IDH mutant −1.546 if MGMT methylated. In some embodiments, the prognostic index score or GPI score is calculated by measuring the expression levels of one or more of the following biomarkers consisting of WIF1, IGFBP3, NGFR, IBSP, HISTLH3G, and COL1A2. In some embodiments, the prognostic index score or GPI score is calculated by measuring the expression levels of two biomarkers. In some embodiments, the prognostic index score or GPI score is calculated by measuring the expression levels of three biomarkers. In some embodiments, the prognostic index score or GPI score is calculated by measuring the expression levels of four biomarkers. In some embodiments, the prognostic index score or GPI score is calculated by measuring the expression levels of five biomarkers. In some embodiments, the prognostic index score or GPI score is calculated by measuring the expression levels of six biomarkers. In some embodiments, the prognostic index score or GPI score is calculated by measuring the expression levels of more than six biomarkers. In some embodiments, the prognostic index score or GPI score is calculated by measuring the expression levels 1, 2, 3, 4, 5, 6, or more biomarkers.

In some embodiments, the present invention may also feature a method comprising obtaining a biological sample from a patient and determining a prognostic index. In some embodiments, a prognostic index is determined by first measuring expression level of one or more of the following biomarkers WIF1, IGFBP3, NGFR, IBSP, HISTLH3G, and COL1A2 and secondly by evaluating an isocitrate dehydrogenase 1 (IDH) mutation status and the MGMT promoter methylation status in a biological sample from said patient. In some embodiments, the prognostic index comprising age of said patient, said measured expression level of one or more of the afromention genes (i.e. WIF1, IGFBP3, NGFR, IBSP, TP53, HISTLH3G, and COL1A2), and the IDH mutation status as well as MGMT promoter methylation status. The prognostic index predicts differential overall survival of said patient. In some embodiments, the method further comprises analysing the prognostic index to determine the patient's prognosis.

### Example

The following is a non-limiting example of the present invention. It is to be understood that said example is not intended to limit the present invention in any way. Equivalents or substitutes are within the scope of the present invention.

GBM Patient, Tumor, and Treatment Characteristics

Clinical Study Design, Patient Selection, and Clinical Data Collection: This study included 24 patients (study group) with a diagnosis of glioblastoma (GBM), who underwent maximal safe resection and completed conventionally fractionated adjuvant radiation (60 Gy) with concurrent TMZ chemotherapy at our institution. A retrospective review of these patients' medical records was conducted to collect de-identified information on their demographics, treatment and tumor characteristics, and treatment outcomes. Overall survival (OS) was defined as the elapsed time between the date of the surgery that established the diagnosis and the date of death. For patients who did not have an IDH1 mutation or MGMT methylation status assayed at the time of diagnosis, their samples were sent to the Clinical Laboratory Improvement Amendments (CLIA) certified reference lab at the Mayo Clinic (Rochester, Minn.) for analysis of these biomarkers. For patients who were still alive at the time of clinical data collection, the date of their last physician encounter or their last imaging procedure, whichever came later, was used to calculate OS. Tumor size (in square centimeters) was defined by multiplying the maximal axial anterior-posterior dimension by the maximal orthogonal lateral dimension on pre-operative contrast-enhanced T1 MRI. The extent of tumor resection was defined as either gross total resection (GTR) or subtotal resection (STR), based on assessment of post-operative MRI by board-certified neuroradiologists. and Karnofsky Performance Score (KPS) groups were defined as either >70 or <70. As a control, archived non-neoplastic cerebral cortical tissue obtained from 12 patients who underwent temporal lobe resection as part of their treatment for epilepsy was utilized. Lateral temporal cortical tissue was obtained and stored as previously described in Fiala et. al. (Fiala M, Avagyan H, Merino J J, et al. Chemotactic and mitogenic stimuli of neuronal apoptosis in patients with medically intractable temporal lobe epilepsy. Pathophysiology. 2013). No clinical information was obtained from this cohort of patients. Formalin-fixed, paraffin embedded (FFPE) GBM tissue or frozen non-neoplastic tissue was unarchived and processed by our institutional Tissue Acquisition and Cellular/Molecular Analysis Shared Resource (TACMASR). This retrospective study was deemed to meet the criteria for exemption under 45 CFR 46.101(b) by the Institutional Review Board (IRB) of the University of Arizona Office for Research and Discovery, Human Subjects Protection Program. This decision was filed under protocol #1709802216.

Median OS of the 24 GBM patients in our institutional study group was 12.3 months. These patients were stratified in two groups: a ‘Short OS’ group, which included patients with less than the cohort's median OS, and a ‘Long OS’ group, with patients whose OS was greater than or equal to the cohort's median survival. A comparison of the demographic, tumor, and treatment characteristics between the ‘Long OS’ and ‘Short OS’ groups are shown in Table 1. No significant differences in baseline characteristics were found between the two groups. The median age of the entire cohort was 63.5 years. There were more males (18) than females (6) in the entire cohort, but the gender proportions between the two groups were identical. Patients in the ‘Long OS’ group trended younger (median age=63 vs 71), with a higher proportion having undergone a GTR (41.7% vs. 16.7%), but these differences were not statistically significant (p=0.16 and 0.37, respectively; Table 1). There was 1 patient with an IDH1 mutation in the ‘Long OS’ group and 1 patient in the ‘Short OS’ group with an indeterminate IDH1 status due to partial staining. There were 4 patients in the ‘Long OS’ group with MGMT promoter methylation. Within the ‘Short OS’ group there were 2 patients with MGMT promoter methylation and 2 with indeterminate MGMT status. The actual mRNA expression of the MGMT gene was also not statistically different between the 2 groups (DE=0.7, p=0.16).

TABLE 1
Patient, Tumor, and Treatment Characteristics

| | Short OS | Long OS | Comparison |
|---|---|---|---|
| | (<=median) | (>median) | p-value |
| Number of Patients | 12 | 12 | |
| Males/Females | 9/3 | 9/3 | 1 |
| Median Age at Dx | 71 (41-80) | 63 (43-77) | 0.16 |
| (Range) | | | |
| Median KPS (Range) | 85 (60-90) | 80 (50-90) | 0.38 |
| Avg Tumor Size in | 21.3 (5.4-36.2) | 22.9 (1.9-41.2) | 0.85 |
| cm2 (Range) | | | |
| Extent of Surgery | | | |
| (% of pts) | | | |
| GTR | 16.7 | 41.7 | 0.37 |
| STR | 83.3 | 58.3 | 0.37 |
| Completion of | 91.7 | 100 | 1 |
| concurrent | | | |
| TMZ (% of pts) | | | |
| Range of RT Dose | 60-74 | 60-75 | |
| (Gy) | | | |

Differential Expression (fold change: Long vs. Short OS)

| | | |
|---|---|---|
| IDH1 | 1.05 | 0.52 |
| MGMT | 0.7 | 0.16 |
| TP53 | 0.94 | 0.6 |
| ATRX | 0.88 | 0.29 |
| PTEN | −0.167 | 0.41 |
| EGFR | 1.22 | 0.11 |

Differential Expression of Pathways and Individual Genes

Next, gene expression profiles were compared between the 24 GBM patients and the 12 temporal lobe tissue samples obtained as part of a treatment of epilepsy that were used as non-neoplastic control. Differential expression (DE) analysis of individual genes identified 326 genes with >2-fold DE and 25 genes with >16-fold DE in the GBM vs non-neoplastic tissue comparison (all adjusted p-values <0.01). Of those genes with >16-fold DE, WIF1 was the only downregulated gene, whereas the other 24 genes displayed increased expression in GBM tissue relative to the non-neoplastic control. In addition, a comparison was made between GBM patients in the ‘Long OS’ group and the ‘Short OS’ group and found that 19 genes displayed >2-fold DE (adjusted p-values <0.05). Cross-referencing the GBM vs non-neoplastic with the ‘Long OS’ vs ‘Short OS’ DE profiles, identified 14 genes, shown in Table 2, which were common to both comparisons.

TABLE 2
Differential Expression of Individual Genes in Our Institutional
Study Group: Genes with a >2-fold change in expression,
with a p value <0.05, in both the non-neoplastic control vs
GBM comparison and the Long OS vs Short OS GBM
comparisons are shown.

| | Log2 Fold | | Log2 Fold | | |
|---|---|---|---|---|---|
| | Change: | | Change: | | |
| | Non- | | GBM | | |
| | Neoplastic | Adj p | Long OS vs | Adj p | |
| Gene | vs Neoplastic | Value | Short OS | Value | Pathway |
| WIF1 | −4.14 | 3.76E−08 | −1.83 | 0.0228 | Wnt |
| CACNA2D3 | −2.3 | 0.000729 | −1.9 | 0.0266 | MAPK |
| WNT7B | −1.38 | 0.00504 | −2.86 | 0.000825 | Hedgehog, |
| | | | | | Wnt |
| PITX2 | −1.37 | 0.00121 | −1.3 | 0.00925 | TGF-beta |
| BMP2 | 1.56 | 3.32E−05 | −1.36 | 0.000992 | Hedgehog, |
| | | | | | TGF-beta |
| MMP7 | 1.78 | 0.000801 | 1.76 | 0.00471 | Wnt |
| PTTG2 | 1.89 | 6.52E−06 | 1.13 | 0.0178 | Cell Cycle- |
| | | | | | Apoptosis |
| BCL2A1 | 1.98 | 0.000468 | 1.11 | 0.0213 | Transcriptional |
| | | | | | Misregulation |
| COL4A6 | 2.72 | 1.41E−06 | −1.18 | 0.0229 | PI3K |
| NGFR | 3.83 | 3.07E−08 | 1.97 | 0.00369 | PI3K, Ras, |
| | | | | | Transcriptional |
| | | | | | Misregulation |
| IGFBP3 | 3.87 | 3.64E−11 | 1.8 | 0.000519 | Transcriptional |
| | | | | | Misregulation |
| COL1A2 | 7.04 | 4.00E−12 | 2.68 | 0.00279 | P13K |
| IBSP | 7.8 | 1.54E−12 | 1.89 | 0.0163 | PI3K |
| HIST1H3G | 8.26 | 2.06E−16 | 1.42 | 0.0149 | Transcriptional |
| | | | | | Misregulation |

A significant dysregulation of the 13 pathways defined by the nCounter PanCancer Human Pathways Panel was also found, a global overview is presented in the form of heatmaps of pathway scores shown in FIGS. 1A-1D. The first panel (FIG. 1A) shows the comparison of pathway scores between the non-neoplastic brain tissue and all GBM tissues, while the second (FIG. 1B) shows the Long versus Short OS comparison for only the GBM samples in the study group. These pathway scores are then summarized as a single pathway signature for each comparison group (FIGS. 1C-1D). In addition to the pathway signatures, the directed global significance scores also provide a global summary measure of the overall differential expression of each pathway, and those are shown in Table 3. The heatmap of GBM versus non-neoplastic brain tissue (FIG. 1A) clearly shows a homogenous expression profile amongst the control brain tissues with consistent patterns of upregulation in about half of the pathways and downregulation in the other half of the pathways when the GBM tissues are compared to those controls. Most notably, the Wnt and MAP kinase pathways were found to be the most negatively dysregulated pathways in the comparison of GBM tissue versus the non-neoplastic controls, while the Notch pathway and genes involved in Transcription Regulation and DNA Damage Repair were strongly positively dysregulated. The same trends, with more heterogeneity, were also observed for these pathways between the Long vs Short OS GBM study group, suggesting a continuum of dysregulation that is associated with outcome. There were two exceptions to this continuum of dysregulation, the Notch and TGF-beta pathways. The Notch pathway was highly upregulated when comparing GBM vs non-neoplastic brain tissue, but was downregulated in the Long vs Short comparison, while the opposite observation was true for the TGF-beta pathway.

TABLE 3
Global Significance Scores

| | Directed Global | Directed Global |
|---|---|---|
| | Significance Score: | Significance |
| | Non-Neoplastic | Score: GBM with |
| Pathways | vs GBM | Long vs Short OS |
| Wnt | −3.145 | −0.867 |
| MAPK | −2.788 | −0.701 |
| Chromatin | −2.694 | −1.141 |
| Modification | | |
| Ras | −2.545 | −0.656 |
| Hedgehog | 0.722 | −1.016 |
| TGF-beta | 1.084 | −1.13 |
| PI3K | 4.159 | −0.351 |
| Driver Gene | 4.218 | −0.474 |
| JAK-STAT | 4.23 | −0.764 |
| Cell Cycle - | 4.724 | −0.358 |
| Apoptosis | | |
| DNA Damage | 4.819 | 0.814 |
| Repair | | |
| Transcriptional | 5.94 | 0.867 |
| Misregulation | | |
| Notch | 6.013 | −1.038 |

Cox Proportional Hazards Analysis and Derivation of Glioblastoma Prognostic Index

Among the 14 genes identified in the combined DE analysis, the 6 genes that had both more than an 8-fold DE in the GBM vs non-neoplastic controls and had more than a 3-fold DE in the ‘Short OS’ vs ‘Long OS’ comparison were focused on. These six genes were WIF1, NGFR, IGFBP3, COL1A2, IBSP, and HIST1H3G. Univariate CPH analysis confirmed that all 6 differentially expressed genes correlated with OS (Table 4). We also performed univariate CPH models for well-known clinical prognostic factors like age, and Karnofsky Performance Score (KPS), tumor size, extent of surgical resection, IDH1 mutation status, and MGMT promoter methylation status, but only age was found to be significantly associated with OS in this small group of patients (Table 4). Next, a multivariate CPH analysis was performed with the factors that were significantly associated with OS on univariate analysis (age and the 6 highly differentially expressed genes), as well as IDH1 and MGMT status because of their well-known significant prognostic implications, despite these factors not being statistically significantly associated with OS in the present study's specific cohort. The regression coefficients obtained from this multivariate CPH model (shown in the right-most column of Table 4) were then used to create a weighted sum of each factor included in the multivariate CPH to create a ‘Glioblastoma Prognostic Index’ (GPI=0.022*Age−0.039*HIST1H3G+0.379*IBSP+0.489*COLIA2+0.046*IGFBP3+0.641*NGFR−0.039*WIF1−2.159 if IDH mutant −1.546 if MGMT methylated).

TABLE 4
Cox Proportional Hazards (CPH) Models: Cox proportional
hazards analysis of the clinical factors commonly associated
with overall survival in GBM patients as well as the 6 genes
that had more than 8-fold DE in the GBM vs non-neoplastic
comparison and more than 3-fold DE in the ‘Short OS’ vs‘
Long OS’ comparison. The regression coefficients shown in
the right column were then used to generate the GPI.

| | | | Multivariate |
|---|---|---|---|
| Univariate CPH Models | | | Regression |
| Factor | HR | p value | Coefficients |
| Age | 1.07 | 0.039 | 0.022 |
| KPS Group (<70 vs >=70) | 1.03 | 0.970 | |
| Tumor Size (cm²) | 1.00 | 0.988 | |
| Extent of Resection | 2.13 | 0.160 | |
| (STR vs GTR) | | | |
| IDH1 Status (WT vs Mutant) | 1.45 | 0.363 | −2.159 |
| MGMT Promoter Status | 0.36 | 0.115 | −1.546 |
| (Methylated vs | | | |
| Unmethylated) | | | |
| Expression Levels | | | |
| HIST1H3G | 1.60 | 0.027 | −0.039 |
| IBSP | 1.32 | 0.045 | 0.379 |
| COL1A2 | 1.44 | 0.008 | 0.489 |
| IGFBP3 | 1.72 | 0.003 | 0.046 |
| NGFR | 1.43 | 0.027 | 0.641 |
| WIF1 | 0.76 | 0.038 | −0.039 |

GPI Survival Analysis for the Study Group

The entire cohort of 24 GBM patients was then stratified into two groups of patients, one group with their GPI below the entire cohort's median GPI Row GPI′ group) and one group with their GPI above the median (‘High GPI’ group). Kaplan-Meier (KM) analysis was used to compare the OS probability between the tow GPI′ and ‘High GPI’ groups. While the median OS for the entire cohort was 12.3 months, the ‘High GPI’ group was found to have a median OS of 7.3 months, significantly shorter than the low GPI′ group, which had a median OS almost 3 times greater at 21.7 months (log-rank p value=0.00004) (FIG. 2A). This methodology is significantly better than using MGMT methylation status alone which resulted in a non-significant difference in OS estimates (18.7 months vs 11.2 months; log-rank p=0.1).

Validation of the GPI in the TCGA

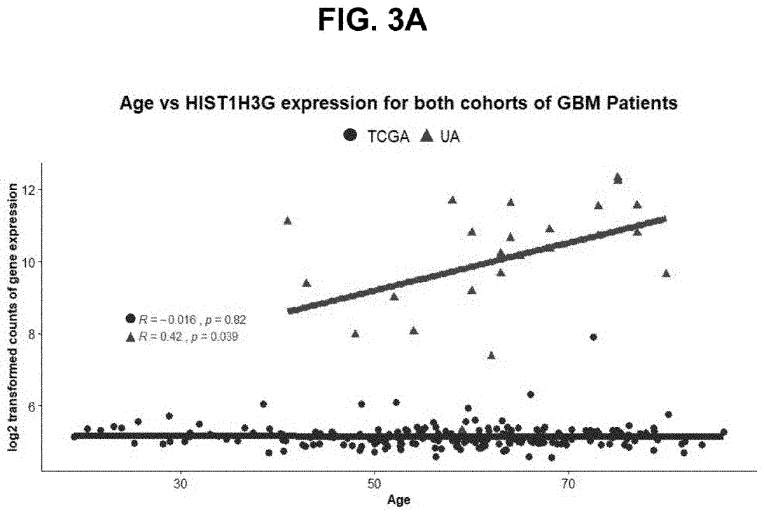

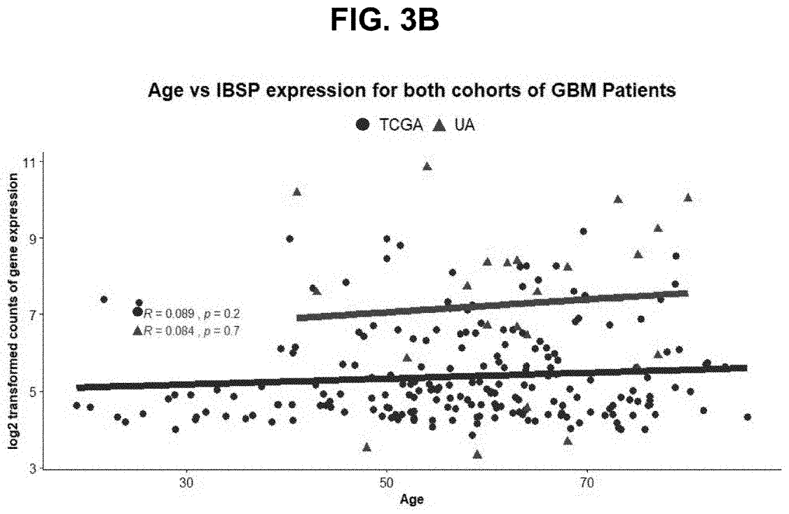

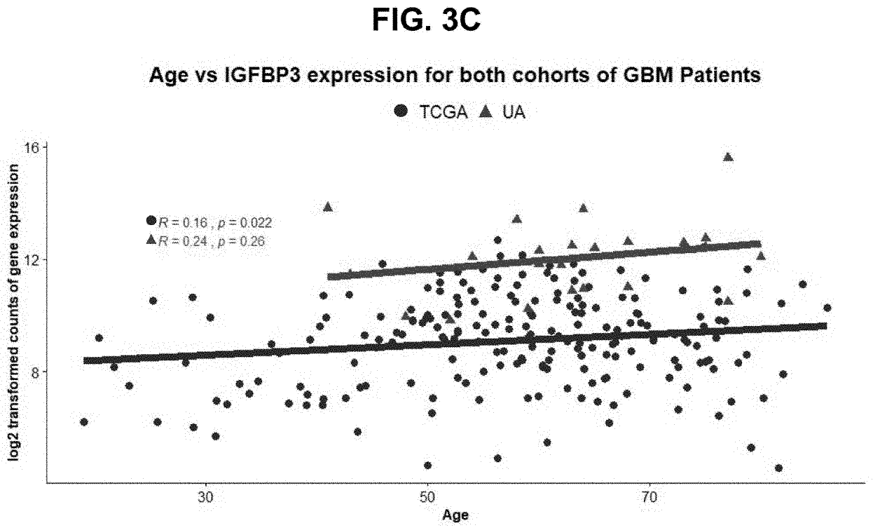

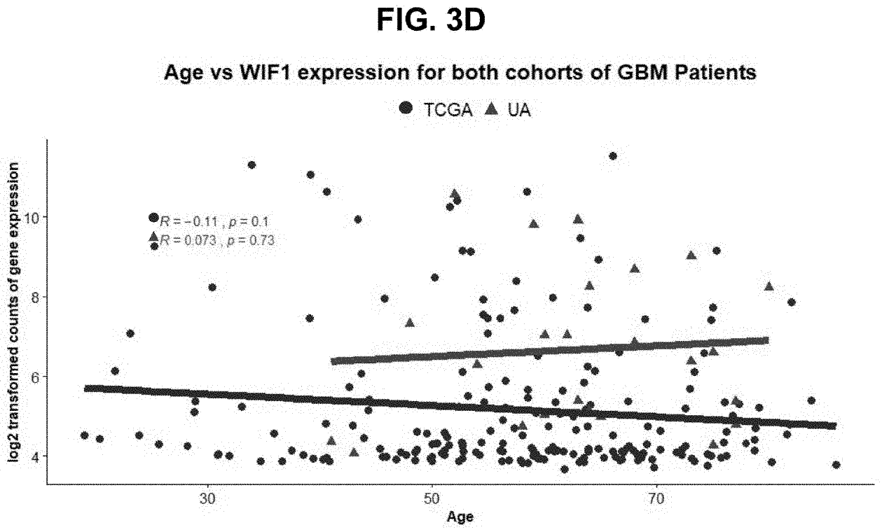

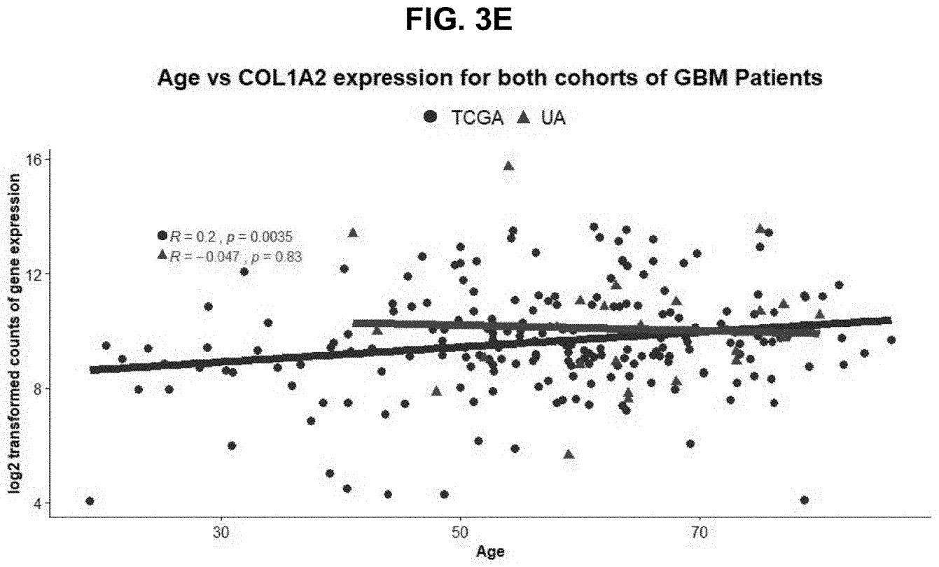

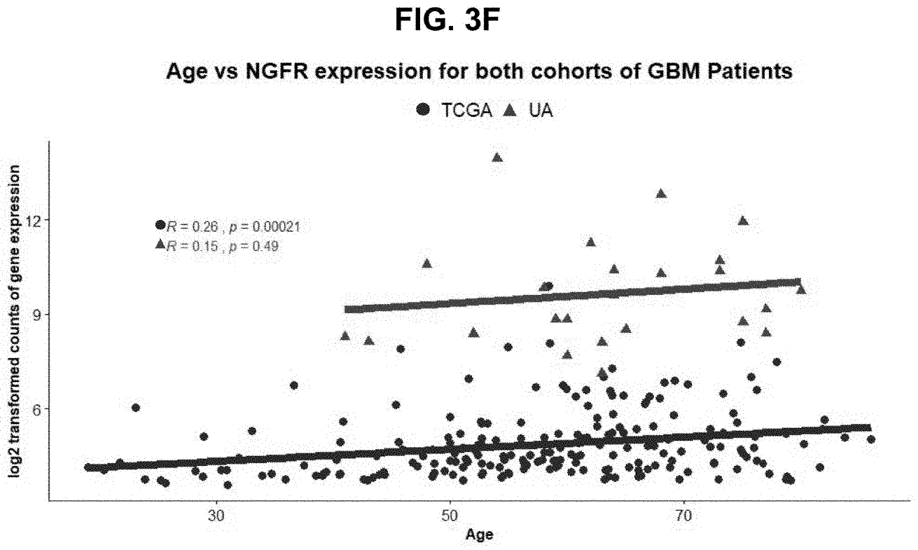

A comparison of the clinical characteristics between the original 24 study group patients and the TOGA cohort used for external validation of the GPI is shown in Table 5. There were no statistically significant differences between the two groups except that the TOGA cohort was statistically significantly younger than the original NanoString cohort (median age of 58.7 versus 63.5; p=0.04). The median OS of the study group cohort was 12.3 months and the median OS for the TOGA cohort was 11.3 months, a difference that was not statistically significant (log rank p=0.5). Because the TOGA cohort of patients was statistically significantly younger than our cohort, we investigated the correlation between age at diagnosis and the expression levels of the 6 genes included within the GPI in both cohorts of patients (FIGS. 3A-3F). The only gene with statistically significant correlation with age within our cohort of 24 patients was HIST1H3G (R=0.42, p=0.04; FIG. 3A), while in the TOGA cohort, COL1A2 (R=0.2, p=0.004; FIG. 3F), IGFBP3 (R=0.16, p=0.02; FIG. 3H), and NGFR (R=0.26, p=0.0002; FIG. 3J) were correlated with age. What is also clear from FIGS. 3A-3F is that the dynamic range of mRNA counts varied significantly between the NanoString quantified cohort (present study) and the microarray quantified TOGA cohort. To account for these differences, the gene expression levels of each cohort were normalized using a Z-score transformation and a new Z-score based multivariate CPH model was calculated for the present cohort to derive the regression coefficients needed to generate a comparable GPI for the 239 TOGA patients. As was done for the study group, TOGA patients were then classified into two risk groups based on their Z-score GPI and compared their KM OS estimates. FIG. 2B shows that within the TOGA cohort of patients, the GPI was also able to distinguish between patients with statistically significant differences in OS. TOGA patients with a high GPI had a median OS of 9.2 months with an estimated 3-year OS of 2.0% while patients with a low GPI had median OS of 12.2 months and a 22.0% 3-yr OS (log rank p value=0.000001). As was seen in the present study group, the separation of survival expectations within the TOGA is greater when using the GPI as compared to using MGMT methylation status alone (median OS=11.2 months when methylated vs 10.4 months for unmethylated; log-rank p=0.04).

TABLE 5
Comparison of the Institutional Cohort and TCGA Cohort

| | NanoString | TCGA |
|---|---|---|
| | Cohort | Cohort |
| Number of Patients | 24 | 239 |
| Males/Females | 18/6 | 144/95 |
| Median Age at Dx (Range) | 63.5 (41-80) | 58.7* (19-86) |
| Median KPS (Range) | 80 (50-90) | 80 (20-100) |
| Avg Tumor Size in cm2 (Range) | 21.2 (1.9-41.2) | unknown |
| Median OS (Months) | 12.3 | 11.3 |
| IDH1 Mutation Status (n/%) | | |
| Wildtype | 22/91.7% | 214/89.5% |
| Mutant | 1/4.2% | 25/10.5% |
| Indeterminate | 1/4.2% | 0 |
| MGMT Promoter Methylation | | |
| Status (n/%) | | |
| Unmethylated | 16/66.7% | 121/50.6% |
| Methylated | 6/25% | 118/49.4% |
| Indeterminate | 2/8.3% | 0 |
| Extent of Surgery (n/%) | | |
| GTR | 7/29.2% | unknown |
| STR | 17/70.8% | |
| Completion of concurrent | 95.8 | |
| TMZ (% of pts) | | |
| Range of RT Dose (Gy) | 60-75 | |

*TCGA Cohort is statistically significantly younger (p = 0.04)

Prognostic Significance of Individual Genes within the GPI

In order to delve deeper into the biological significance of each gene included within the GPI, the prognostic significance was then analyzed of each gene individually. To do this each cohort of GBM patients was stratified into two groups, with patients defined as having either High or Low expression of each gene if their individual expression was above or below the median expression for their cohort. KM comparison of each of these groups then identified 2 genes, IGFBP3 and NGFR, with significantly different OS probabilities using this simplified binary (high/low) classification system in both our study group and in the TOGA validation cohort. The median OS for patients with high IGFBP3 levels was 9.7 months in the present cohort and 10.3 months in the TOGA cohort. In contrast, patients with low levels of IGFBP3 had a median OS of 17.6 months in the present cohort (log-rank p-value being 0.01; FIG. 2I) and 12.5 months in the TOGA (log-rank p=0.0004; FIG. 2J). In the case of NGFR, patients with high levels had a median OS of 7.8 months in the present cohort and 9.2 months in the TOGA versus 18.2 months and 12.2 months in the present cohort and the TOGA cohort respectively (log-rank p=0.05, FIG. 2K for our study group; log-rank p=0.02, FIG. 2L for the TOGA).

There were 2 genes, HIST1H3G and WIF1, with different OS estimates in the present study group but not within the TOGA. In the present cohort, the HIST1H3G levels inversely correlated with survival as patients with low HIST1H3G levels had a median OS of 20.9 months versus 8.6 months for those with high HIST1H3G levels (log rank p value=0.01; FIG. 2C). In contrast, WIF1 levels positively correlated with survival as patients with high WIF1 expression had a median OS of 17.9 months compared to 10.6 months for patients with low expression (log rank p value=0.05; FIG. 3M). Finally, there was one gene, COL1A2, that had significantly different OS in the TOGA cohort but not within the present study group. Within the TOGA cohort, patients with low expression levels of COL1A2 had a median OS of 11.5 months versus 10.1 months for patients with high expression levels (log rank p value=0.002; FIG. 2H).

From this small retrospective study of 24 GBM patients uniformly treated with standard of care, the present invention has derived a GBM prognostic index (GPI), based on patient age and the expression levels of 6 genes, that can significantly refine OS prediction in GBM patients undergoing treatment with the current standard of care. The prognostic power of this index is demonstrated by the fact that the median OS of patients with a low GPI was found to be nearly three-times that of patients with a high GPI (21.7 months vs. 7.3 months; FIG. 3A). The significance of this GPI was then validated with independent gene expression data from a larger group of patients from TOGA that again showed clinically meaningful differences in the OS of patients based on their GPI (23.3% vs 3.1% 3-yr OS). The usefulness of the GPI is in its ability to refine our prognostication abilities which in turn would significantly improve the patient and physician decision-making process regarding optimal adjuvant treatment after maximal safe resection. For instance, patients with a high GPI could elect for a less burdensome, hypofractionated radiation treatment course, such as those that have already been shown to be non-inferior to the standard Stupp-based regimen in subsets of patients with poor prognosis or patients with a low GPI could elect to enroll in more aggressive experimental treatment protocols instead of the standard regimen.

In summary, the work reported here has defined a panel of clinical and genomic factors that significantly refines OS prognosis in GBM patients. In addition, the highly dysregulated nature of these genes and their correlations with patient survival identifies some of these altered genes (NGFR, IGFBP3, and potentially WIF1 and HIST1H3G) as potential therapeutic targets for GBM. While these results are intriguing, they need to be confirmed in larger clinical trials in order to validate the GPI gene expression panel as a potential prognostic tool in GBM.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method for treating a patient in need thereof who has a condition that causes or is caused by dysfunction of the Wnt signaling pathway, said method comprising:
a) obtaining a biological sample from the patient;
b) determining the prognostic index of the patient by:
i) measuring expression level of one or more of the following biomarkers WIF1, IGFBP3, NGFR, IBSP, HISTLH3G, and COL1A2 in the biological sample from said patient; and
ii) evaluating an isocitrate dehydrogenase 1 (IDH) mutation status and the MGMT (O6-Methylguanine-DNA Methyltransferase) promoter methylation status;
wherein the prognostic index comprises the age of said patient, said measured expression level of one or more WIF1, IGFBP3, NGFR, IBSP, TP53, HISTLH3G, and COL1A2 from (i), said IDH mutation status and MGMT promoter methylation status from (ii) wherein said prognostic index predicts differential overall survival of said patient; and
c) administering a therapeutically effective amount of drug or intervention to said patient to treat said condition based on said prognostic index.

2. The method of claim 1, wherein the condition that causes or caused by dysfunction of Wnt signaling comprises Glioblastoma Multiforme (GBM), cancers that are radio-resistant or eligible to receive radiation therapy, or any condition that causes or is caused by Wnt signaling or insulin-like growth factor signalling dysfunction, or a condition with dysregulation in signaling pathways comprising NGFR, IBSP, and or TP53.

3. The method of claim 1, wherein the biological sample obtained from said patient comprises tissue, cerebrospinal fluid, and/or blood.

4. The method of claim 1, wherein said expression further comprise FZD9, WNT7B, SFRP1, FZD7, PRKCG, PRKCB, PPP3CB, CCND1, TP53, COL4A6, MMP9, MMP7 or a combination thereof.

5. The method of any one of claim 1, wherein said expression level comprises protein expression level, gene expression level or mRNA levels, DNA levels, and/or protein activity level.

6. The method of any one of claim 1, wherein measuring said expression level comprises measuring protein expression, measuring gene expression or mRNA levels, measuring DNA levels, and/or measuring protein activity.

7. The method of any one of claim 1, wherein the expression level of WIF1 IGFBP3, NGFR, IBSP, HISTLH3G, and COL1A2 is measured at baseline.

8. The method of claim 7, wherein said baseline expression level is relative baseline level at the time of initial presentation or diagnosis of the condition.

9. The method of claim 7, wherein baseline levels are normal levels from an aggregate population of asymptomatic individuals without the condition.

10. The method of any one of claim 1, where in the prognosis index score is calculated using the gene expression values of this panel of 6 genes, and normalized using appropriate statistical algorithms, wherein the range of prognosis index scores comprises a range of 0-30 and the cut point between good and poor prognosis groups is either the median prognosis index score of a large cohort of patients, or determined using statistical techniques such as the maximal rank statistics test, or other appropriate statistical techniques.

11. The method of any one of claim 1, wherein the prognosis index is determined with the following formula, 0.022*Age−0.039*HIST1H3G+0.379*IBSP+0.489*COL1A2+0.046*IGFBP3+0.641*NGFR−0.039*WIF1−2.159 if IDH mutant −1.546 if MGMT methylated.

12. The method of claim 1, wherein said therapeutically effective drug or intervention modulates Wnt signaling.

13. The method of claim 12, wherein said therapeutically effective drug or intervention comprises temozolomide, pyrvinium or XAV939, chemical derivatives thereof, radiation, modulators of Wnt signaling, inhibitors to IGFBP3, and/or activators of WIF

14. A method of monitoring effectiveness of a treatment that is currently being administered to a patient in need thereof who has a condition that causes or is caused by dysfunction of the Wnt signaling pathway, said method comprising:
a) obtaining a biological sample from the patient;
b) determining the prognostic index of the patient by:
i) measuring expression level of one or more of the following biomarkers WIF1, IGFBP3, NGFR, IBSP, HISTLH3G, and COL1A2 in the biological sample from said patient; and
ii) evaluating an isocitrate dehydrogenase 1 (IDH) mutation status and the MGMT (O6-Methylguanine-DNA Methyltransferase) promoter methylation status;
wherein the prognostic index comprises the age of said patient, said measured expression level of one or more WIF1, IGFBP3, NGFR, IBSP, TP53, HISTLH3G, and COL1A2 from (i), said IDH mutation status and MGMT promoter methylation status from (ii) wherein said prognostic index predicts differential overall survival of said patient; and
c) if the patient has a poor prognosis, administering a different treatment to the patient, wherein a poor prognosis is determined when the patient has a shorter overall survival compared to a median overall survival, or if the patient has a good prognosis, maintaining the current treatment of the patient; wherein a good prognosis is determined when the patient has a longer overall survival compared to a median overall survival.

15. The method of claim 14, wherein the condition that causes or caused by dysfunction of Wnt signaling comprises Glioblastoma Multiforme (GBM), cancers that are radio-resistant or eligible to receive radiation therapy, or any condition that causes or is caused by Wnt signaling or insulin-like growth factor signalling dysfunction, or a condition with dysregulation in signaling pathways comprising NGFR, IBSP, and or TP53.

16. The method of claim 14, wherein the biological sample obtained from said patient comprises tissue, cerebrospinal fluid, and/or blood.

17. The method of claim 14, wherein said expression further comprise FZD9, WNT7B, SFRP1, FZD7, PRKCG, PRKCB, PPP3CB, CCND1, TP53, COL4A6, MMP9, MMP7 or a combination thereof.

18. The method of any one of claim 1, where in the prognosis index score is calculated using the gene expression values of this panel of 6 genes, and normalized using appropriate statistical algorithms, wherein the range of prognosis index scores comprises a range of 0-30 and the cut point between good and poor prognosis groups is either the median prognosis index score of a large cohort of patients, or determined using statistical techniques such as the maximal rank statistics test, or other appropriate statistical techniques.

19. The method of any one of claim 1, wherein the prognosis index is determined with the following formula, 0.022*Age−0.039*HIST1H3G+0.379*IBSP+0.489*COL1A2+0.046*IGFBP3+0.641*NGFR−0.039*WIF1−2.159 if IDH mutant −1.546 if MGMT methylated.

20. A method comprising:
a) obtaining a biological sample from a patient;
b) determining a prognostic index by;
i) measuring expression level of one or more of the following biomarkers WIF1, IGFBP3, NGFR, IBSP, HISTLH3G, and COL1A2 in a biological sample from said patient; and
ii) evaluating an isocitrate dehydrogenase 1 (IDH) mutation status and the MGMT promoter methylation status;
wherein the prognostic index comprises age of said patient, said measured expression level of one or more WIF1, IGFBP3, NGFR, IBSP, TP53, HISTLH3G, and COL1A2 from (a), said IDH mutation status and MGMT promoter methylation status from (b);
wherein said prognostic index predicts differential overall survival of said patient;
c) analysing the prognostic index.
